(19) United States
(12) Patent Application Publication
(10) Pub. No.: US 2011/0039277 A1
(43) Pub. Date: Feb. 17, 2011

(54) Methods of Labeling Proteins

(76) Inventors: Pier Mastroberardino, Pittsburgh, PA (US); John Greenamyre, Sewickley, PA (US)

Correspondence Address: Hirshman Law, LLC, Gatehouse Building, 101 W. Station Square Dr., Suite 500, PITTSBURGH, PA, 15219, US

(21) Appl. No.: 12/937,739

(22) PCT Filed: Apr. 17, 2009
(86) PCT No.: PCT/US09/40976

§ 371 Date: Oct. 14, 2010

Related U.S. Application Data
(60) Provisional application No. 61/124,655, filed on Apr. 18, 2008.
(60) Provisional application No. 61/134,902, filed on Jul. 15, 2008.

(51) Int. Cl. G01N 33/53; C07K 1/13; C12N 9/96; G01N 33/566

(52) U.S. Cl. 435/7.1; 530/402; 435/188; 436/501

(57) ABSTRACT

Methods are provided for labeling specific oxidized proteins. Methods also are provided for determining the oxidation state of a cell. Such methods are useful as diagnostic, therapeutic and screening agents.

### CROSS REFERENCE TO RELATED APPLICATIONS

This application is a National Stage of International Application No. PCT/US2009/040976, filed Apr. 17, 2009, which in turn claims the benefit of U.S. Provisional Application Nos. 61/124,655, filed Apr. 18, 2008, and 61/134,902, filed Jul. 15, 2008, each of which is incorporated herein by reference in its entirety.

Thiols are the functional groups in the side chain of the amino acid, cysteine (Cys). Depending upon the oxido-reductive (redox) status of the surrounding environment, thiols exist either in the reduced form of free thiols (—SH) or oxidized to disulfides (—S—S—). Disulfides are well known to play an important structural role, contributing to the maintenance of the proper folding in proteins. More recently it was found that the formation of disulfides in proteins also exerts critical regulatory functions: vital mechanisms such as protein import, regulation of signal transduction cascades, regulation of the activity of transcription factors and proper function of the mitochondrial electron transport system rely on disulfide oxidation or reduction. Moreover, the formation of disulfides represents an early, reversible response to oxidative stress; it precedes higher forms of oxidation, e.g., carbonylation as well as the formation of sulfenic or sulfonic acids. Therefore, disulfide formation may play a protective role.

Cysteine residues control the redox environment through reversible oxidation of thiols to disulfides. As oxidative stress increases, a high number of Cys in the protein thiol pool—the third system of redox regulation with glutathione and thioredoxins—will be oxidized to disulfides. The reversible conversion of thiols to disulfides is one the earliest observable events during radical-mediated oxidation of proteins. Thiol oxidation has functional consequences as it may be correlated to protein inactivation.

Increasing interest in the study of thiols has led to the development of a variety of technical approaches to detect thiols and disulfides. In particular, several spectrophotometry-based protocols were developed to measure thiol levels. More recently, proteomic methods to identify disulfides in proteins have been established. However, means to image exact loci of thiol oxidation in cells and tissues are quite limited

Imaging techniques would be particularly useful, since the redox environment differs between cell types and even subcellularly. In fact, the level of free thiols varies greatly in subcellular compartments. Whereas the endoplasmic reticulum provides a very oxidizing milieu, the cytosol is highly reducing. Also, the intra- and extracellular environments are distinct, with more oxidizing conditions in the extracellular space. Finally, in heterogeneous organs, such as the brain, susceptibility to altered redox potential may differ between cell types because of their different metabolic properties.

The major metabolic processes, such as biosynthethic pathways and oxidative phosphorylation, involve fluxes of electrons between chemical couples: a donor (the oxidized species) and an acceptor (the reduced one). The physical parameter that will determine if the electron flux will happen—and to what extent—is the electromotive potential of the involved chemical species. Flux occurs spontaneously from more negative potential to less negative ones, so electrons travel in a preferential direction. As a direct consequence, the cellular environment has a natural tendency to oxidize. This physiological trend toward oxidation is counterbalanced—and indeed finely tuned—by the enzymatic and non-enzymatic activity of antioxidant species to maintain adequate oxido-reductive (redox) homeostasis. If under any circumstance the redox balance is altered in favor of the pro-oxidant events, the state is defined as oxidative stress.

Under normal physiological conditions, the control of the redox homeostasis relies mostly on the thiol groups of cysteine (Cys) residues. These residues are particularly suited to this task in that—upon variations of the redox environment—they can be readily oxidized to disulfides. This is a one-electron exchange reaction, which is reversible and non-enzymatic. It constitutes an early response to buffer alterations in the redox homeostasis. Three independent major mechanisms participate in cellular redox control; all of them are based on the reduction/oxidation of Cys residues. Two mechanisms rely on molecules specialized in controlling the redox state—(i) glutathione (GSH), which is very abundant in the cell and has been considered the principal buffer, and (ii) thioredoxins (TRXs). In association with those two systems, some Cys residues belonging to other proteins or enzymes—which possess other primary function than controlling the redox state—can undergo oxidation/reduction cycles too, therefore participating to redox control. This last set of Cys constitutes the protein thiol pool. Besides contributing to redox regulation, oxidation of Cys in the protein thiol pool can have other important effects:

It can be protective, preventing higher forms of oxidation, such as the formation of sulfenic acid—an irreversible, two-electron oxidation of Cys residues.

It can change the physical properties of the protein, promoting events such as aggregation.

It can alter the function of the protein, either providing new functions (gain of function) or reducing the normal one (loss of function).

While the systems of glutathione and thioredoxins are being actively investigated, the protein thiol pool has not been studied adequately.

In the case of oxidative stress, the increase in the production of reactive oxygen species will generate an abnormal flux of electrons, which will overwhelm the cellular buffering systems—glutathione, thioredoxins and the protein thiol pool. Therefore, a variety of molecular species will be affected. The identity of such affected molecules will depend upon several factors including:

The sites of generation of ROS; different sites are surrounded by different molecular environments. Therefore, a disease or toxin, such as a pesticide generating ROS at the level of respiratory complex I in mitochondria, will influence different molecules than a disease or toxin generating ROS on the plasma membrane.

The concentration of the target molecule; molecules that are highly represented around the site of generation of ROS will have better chances to be oxidized.

The constant defining the rate of attack of the radicals; some molecules are more susceptible to radical attack and electron transfer than others.

Proteins, in general, are ubiquitous, very abundant and possess rate constants for radical attack that are generally higher than in other biomolecules, such as lipids and DNA. Hence, they fully meet the above criteria and constitute excellent targets for oxidative modification. The heterogeneous composition of proteins—due to 20 different amino acids plus unusual ones (such as selenocysteine) and post-translational modifications—allows a great variety of modifications. The susceptibility to oxidation of a certain amino acid residue in a protein will depend on factors such as (1) the functional group in the side chain, (2) the molecular environment of amino acid (e.g., the primary sequence as well as the structure of the protein) and (3) the spatial relationship—proximity and orientation—with respect to the ROS source.

As the oxidative imbalance increases, amino acids other than Cys can be irreversibly oxidized; many of these modifications require more stringent oxidant conditions than disulfide formation. Among the possible modifications, formation of carbonyls—which occurs especially on the side chains of Lys, Arg, Pro and Val—is the most extensively studied. For instance, in the case of toxins, carbonyl formation has been detected in both rotenone and paraquat poisoning. Carbonyls have attracted particular attention because reproducible, well-established experimental assays are available. In fact, carbonyls—which are aldehydes and ketones—can be efficiently derivatized with hydrazine-compounds, which can then be detected by several means, including specific antibodies. Carbonyl formation can be induced by different oxidizing agents and often results in loss of protein function. Carbonyls are relatively difficult to induce compared to cysteine oxidation and, indeed, high carbonyl levels tend to indicate that oxidative stress is already associated with some kind of disease-related dysfunction. Therefore, the study of carbonyl formation has great relevance to understand the mechanistic aspects of pathologies; however, it is believed to constitute a very advanced stage in the cascade of oxidative events and is not ideally suited as an early marker for pathology.

A further set of oxidative modifications that can affect proteins derives from the interaction of nitric oxide (NO) with molecular oxygen to generate peroxynitrite (ONOO—), which, in turn, can react with some side chain amino acids, including tyrosines and cysteines, to form respectively nitrotyrosine and nitrosocysteine. For example, microglial activation is a pronounced event in both rotenone and paraquat poisoning and involves the up-regulation of inducible nitric oxide synthases (iNOS). iNOS produces large amounts of nitric oxide, as well as activation of NADPH oxidases, which reduce oxygen to form superoxide. This sequence of events favors the production of peroxynitrite, which has been strongly implicated in the toxicity of rotenone and paraquat in laboratory animals, as well as in Parkinson's disease (PD) pathogenesis. Oxidation to nitrotyrosine and nitrosocysteine are rather specific and can be reversed by nitrase enzymes.

The etiology of Parkinson's disease (PD) has not been completely elucidated. Recent evidence indicates that the general clinical symptoms of PD—rather than sharing uniform etiology—are the results of different, converging pathogenic pathways. Among the factors responsible for PD, exposure to different pesticides has been widely correlated to the development of the disease. Even though different mechanisms can lead to PD, oxidative damage is a common feature of the disorder as well as a general consequence of pesticide intoxication. Due to their abundance and their ubiquitous presence, proteins are very susceptible to oxidative insult, which can induce post-translational modifications altering the normal function of the protein. Among the several possible modifications, oxidation of thiols to form disulfides represents an early, reversible response to oxidative stress.

Although oxidative stress is an area of active investigation in many fields of biomedical research, no tools have available to image thiol oxidation in proteins in intact tissues. There is great advantage to being able to detect thiol oxidation in histological samples. Many organs have a complex composition of cell types and classical biochemical approaches on total tissue homogenates do not allow discrimination of the particular cell type that is affected. Moreover, under pathological conditions, the relative composition of cell types can vary and the measure of levels of oxidation could be biased by the proliferation of a certain cellular population. An example of this scenario is found in neurodegenerative disorders, where neurons tend to die and glia tend to proliferate.

Diagnosis, treatment and research for neurodegenerative disorders, among others, would benefit greatly from an imaging-based technology that is capable of evaluating thiol redox state in histological preparations. Current spectrophotometric and proteomic techniques only detect redox states of sub-cellular fractions of total tissue homogenates, so they are not suitable to detect differences in the redox state of single cell types in complex tissues. Consequently, new methods of cellular redox state detection and imaging are needed.

### SUMMARY

Methods of labeling proteins in biological samples are provided as well as methods for identifying the oxidative state of a cell. Also, methods for testing a chemotherapeutic agent are provided as well as methods for determining whether an agent can prevent tumor growth.

As such, methods of labeling cells or proteins in a biological sample are provided and include labeling oxidized groups in proteins, such as carbonyl or disulfide groups; determining an oxidative state of proteins in a cell or biological sample; and determining if a compound, such as an antioxidant or a chemotherapeutic agent, can affect the oxidative state in a cell. The cell can be a cultured cell or a cell of an organism, such as a human or animal subject. The cell can be from normal tissue or from tumor tissue samples.

According to one embodiment, a method of labeling a protein target in a biological sample is provided. The method comprises binding the protein with an oxidation labeling reagent, wherein the labeling reagent comprises a first member of a fluorescence resonance energy transfer (FRET) pair and a carbonyl-reactive group. The method may further comprise binding a second labeling reagent comprising a second member of a fluorescence resonance energy transfer (FRET) pair to a protein target in the same biological sample such that the first reagent and the second reagent are in such close proximity as to provide a FRET signal.

The FRET pair can be, without limitation, one of the pairs: CPM/Alexa488 and CPM/FITC and FITC/Cy3. The carbonyl-reactive group can be hydrazine or a hydrazine derivative, such as dinitrophenyl hydrazine, including for example fluorescein-conjugated hydrazide. In certain other embodiments, the second labeling reagent is, for example, an antibody directed to a cellular protein that is coupled to a fluorochrome. FRET may be detected or quantified by one of fluorescent imaging, spectrophotometry, and fluorescent-activated cell sorting, fluorescent microscropy, among others.

A method of detecting the presence of carbonyl groups on proteins in a biological sample is also provided. For example in one method, the protein target is labeled as described herein and the relative FRET between the first and second members of the FRET pair in the sample is measured.

According to another embodiment, a method of labeling oxidized thiols (e.g., disulfides) in a biological sample is provided. The method comprises: alkylating free thiols in the sample with an alkylating agent; reducing disulfide bonds of proteins in the sample with a reducing agent to form free thiols; and labeling the free thiols in the sample with a first member of a FRET pair attached to an alkylating group. The alkylating agent can be, for example, one or more of a haloalkyl compound, an alpha-halocheto compound, an N-alkylmaleimide, an alkylmethanethiosulfonate, a p-hydroxymercurybenzoate, Ellman's reagent and a metal ion. In one embodiment, the alkylating agent is a combination of iodoacetamide and N-ethylmaleimide. The alkylating agent can be one or more of monobromobimane, iodoacetic acid, iodoacetamide, N-ethylmaleimide, and methyl methanethiosulfonate, and mercury orange. The alkylating group can be, for example, one of a haloalkyl compound, an alpha-halocheto compound, a N-alkylmaleimide, an alkylmethanethiosulfonate, a p-hydroxymercurybenzoate, Ellman's reagent and a metal ion. For example, one of monobromobimane, iodoacetic acid, iodoacetamide, N-ethylmaleimide, and methyl methanethiosulfonate, and mercury orange. In some instances, the alkylating group can serve as a member of a FRET pair, and can be described as an alkylating agent that is a fluorophore, such as monobromobimane and mercury orange. The reducing agent can be chosen from, for example, one of Cleland's reagent, DTT (dithiothreitol) or DTE (dithioerythriol); 2-mercaptoethanol (β-mercaptoethanol); 2-mercaptoethylamine; and trivalent phosphines, such as TBP (tributylphosphine) or TCEP (tris(2-carboxyethyl)phosphine).

Also provided is a method detecting the presence of oxidized thiol groups of proteins in a biological sample, comprising labeling proteins in the biological sample and binding a binding reagent to a protein target as described above and detecting and/or quantifying FRET between the first and second members of the FRET pair in the sample. The FRET pair can be, without limitation, one of the pairs: CPM/Alexa488 and CPM/FITC and FITC/Cy3.

According to yet another embodiment, a method of labeling oxidized thiols (e.g., disulfides) in a biological sample is provided. The method comprises: alkylating free thiols in the sample with an alkylating agent; reducing disulfide bonds of proteins in the sample with a reducing agent to form free thiols; and labeling the free thiols in the sample with a dye. The dye can have an emission spectrum, without limitation, in the ultraviolet, near-ultraviolet, visible, near-infrared, or infrared spectra.

In yet another embodiment, a method of identifying a disease or exposure to a toxic compound in a subject, such as a human or animal is provided. The method comprises obtaining a biological sample from a subject and determining the oxidation state in the biological sample by any of the methods described herein. The results of such an assay can provide an indication of pathology.

In another embodiment, a method of determining if a compound can restore a cell from abnormal cellular oxidation state is provided. The method comprises: contacting one or more cells having an abnormal cellular oxidation state with a compound of interest; and detecting and/or quantifying in the cell the presence of oxidized thiol or carbonyl groups as compared to a control which is not contacted with the compound. An abnormal oxidation state may be caused by a pathology such as disease, cancer, genetic defect and/or a toxin. The toxin may be a pesticide, such as rotenone or paraquat. The compound tested may be an antioxidant.

In another embodiment, a method of determining if an agent (e.g., a compound, composition, drug, molecule, etc.) can affect the cellular oxidation state in a tumor cell is provided. Such methods comprise: contacting one or more tumor cells with one or more chemotherapeutic agents; detecting and/or quantifying the presence of oxidized thiols or carbonyls in the tumor cell; and comparing the oxidation level of the cells treated with the chemotherapeutic agent(s) to a control, wherein increased oxidation level in the tumor cells treated with the chemotherapeutic agent(s) is indicative of the ability of the agent(s) to induce oxidation of thiols and thereby prevent tumor growth. Thus, in a related embodiment, a method of determining if an agent can prevent tumor growth is provided. The tumor cells can be obtained from any organ or tissue, such as, skin, liver, or spleen. The chemotherapeutic agent tested may be a platinum-based chemotherapy drug, an antibiotic, an antioxidant, alkylating agent, anti-metabolite, cytokine, and/or a free radical scavenger.

Tumor cells can be obtained from any organ or tissue, such as, skin, liver, or spleen. The chemotherapeutic agent tested may be a platinum-based chemotherapy drug, an antibiotic, an antioxidant, alkylating agent, antimetabolite, cytokine, and/or a free radical scavenger.

In yet another embodiment, a kit is provided. The kit comprises packaging, as is commercially acceptable and contains: a first container having an alkylating agent, a second container having a reducing agent and a third container having a first member of a FRET pair attached to an alkylating group. The kit may further comprise a fourth container having a binding reagent conjugated to a second member of the FRET pair. As automated sample processing systems are becoming more prevalent, the kit may comprise a cartridge suitable for use in such an automated sample processing system, such that the first, second, third, and optionally fourth containers are elements configured within a cartridge for use in an automated sample processing system. The alkylating agent, reducing agent, first member of a FRET pair attached to an alkylating group, and binding reagent conjugated to a second member of the FRET pair include, without limitation, all examples of such reagents provided herein.

In another embodiment, the kit comprises packaging, as is commercially acceptable and comprises a first container having a labeling reagent, the labeling reagent comprising a first member of a fluorescence resonance energy transfer (FRET) pair conjugated to a carbonyl-reactive group. The kit may further comprise a second container having a binding reagent conjugated to a second member of the FRET pair. The kit may comprise a cartridge suitable for use in an automated sample processing system, such that the first, and optionally second containers are elements configured within a cartridge for use in an automated sample processing system. The labeling reagent and binding reagent conjugated to a second member of the FRET pair include, without limitation, all examples of such reagents provided herein.

### BRIEF DESCRIPTION OF THE DRAWINGS

FIG. 1A is a schematic of the reaction of DNPH (dinitrophenylhydrazine) with carbonyls. Hydrazide is the functional group responsible for the reaction (highlighted in yellow).

FIG. 1B is a schematic of fluorescein-conjugated hydrazine. The reactive group (in yellow) is identical to the DNPH functional group. The fluorophore is highlighted in green.

FIG. 2 shows fluorescence microphotographs of carbonyl labeling in vehicle-treated and rotenone-treated cells. Twenty four hours exposure to 50 nM rotenone resulted in a detectable increase in green fluorescence (carbonyls). Glyceraldehyde 3-phosphate dehydrogenase (GADPH) is a “housekeeping” protein normally expressed in cells (red).

FIG. 3-2 shows fluorescence microphotographs of FRET detection with the acceptor photobleaching method. In rotenone-treated cells (lower panel), disruption of Cy3 (red), the acceptor fluorophore, induces a significant increase in FITC emission (green). Such results demonstrating that the two fluorophores are in close proximity indicated that the protein of interest, Hsp60, is oxidized.

FIG. 4 is a graph depicting staining of disulfides (blue line), free thiols (red lines) and carbonyls (green line) in a 96 well plate assay.

FIG. 5 shows schematics of the general experimental strategy used to label thiols in cells and tissues. The left panel outlines one embodiment of the method for labeling thiols in cells. In brief, after blocking free thiols with specific alkylating agents and washing the cells extensively to remove the unreacted alkylating agents, the thiols that were previously engaged in disulfides are reduced and labeled with a specific probe. The right panel shows the chemical reaction for one embodiment of the method for labeling thiols in cells. In most cases, free thiols were blocked with NEM-IAA (N-ethylmaleimide-iodoacetamide) and disulfides were reduced with TCEP (tris(2-carboxyethyl)phosphine)). Subsequently, exposed thiols were labeled with CPM (7-diethylamino-3-(4′-maleimidylphenyl)-4-methylcoumarin). An important negative control consists of omitting the reducing step.

FIG. 6 are SDS gels showing the identification of the best UV-probe to label thiols in proteins retaining their native conformations. After mild permeabilization of cells with digitonin, protein-thiols were labeled with UV-probes containing different functional groups. All the probes were used 0.5 mM final. (a) Proteins were separated by SDS electrophoresis and the gel was imaged with a UV transilluminator. CPM (lane 3) was the most effective probe and was used for the subsequent experiments in this study. (b) Coomassie staining of the same gel ensured equal protein loading. Abbreviations represent: CPM for 7-diethylamino-3-(4′-maleimidylphenyl)-4-methylcoumarin; MIANS for 2-(4′-maleimidylanilino)naphthalene-6-sulfonic acid; IAEDANS for 5-((((2-iodoacetyl)amino)ethyl)amino)naphthalene-1-sulfonic acid; and IAANS for 2-(4′-(iodoacetamido)anilino)naphthalene-6-sulfonic acid.

FIG. 7 are fluorescence microphotographs showing detection of oxidized thiols in cells. Treatment with the pro-oxidant t-butyl hydroperoxide (tBH) (1 mM) induced a remarkable increase in disulfides (f) compared to vehicle control (e). As expected, no signal was detectable in the negative control (d), where the reducing step of the protocol with TCEP was omitted. The confocal microscope scanning parameters were set in the control reaction and kept equal for all the following analyses. Panels (a)-(c) shows labeling with thioredoxin-1 (Trx-1).

FIG. 8 are fluorescence microphotographs showing detection of oxidized cysteines in tissues. Rats were treated chronically with rotenone, a mitochondrial toxin associated with oxidative damage and the development of experimental parkinsonism. Oxidized thiols (blue fluorescence) colocalized extensively with the dopaminergic neuronal marker (TH, red), but not with the glial marker (GFAP, green). This is the first evidence that this type of oxidation occurs mainly in neuronal cells.

FIG. 9 shows the basics of fluorescence energy transfer (FRET) and why CPM and Alexa488 are a good FRET pair. For FRET to occur, spectrally compatible fluorophores must be within about 50 Å of each other. FIG. 9A shows that when the fluorophores are close enough, some of the energy from the excited donor fluorophore is transferred to the acceptor fluorophore. As a result, the direct fluorescence of the donor is reduced and fluorescence of the acceptor can be detected. FIG. 9B shows that the CPM emission spectrum upon excitation at 380 nm overlaps with the excitation spectrum of Alexa488 (shaded area), which is a property that renders the two fluorophores a good FRET pair. FIG. 9C shows the emission spectra demonstrating in vitro that CPM and Alexa488 make a good FRET pair. When the same goat IgG molecule was labeled with both CPM and Alexa488, excitation of CPM induced emission of Alexa488 due to fluorescence energy transfer (gray line).

FIG. 10 shows the detection of oxidized thioredoxin-1 (trx-1) in cells. FIG. 10A is a schematic of the procedure used. FIG. 10B is a Western blot showing that the treatment of cells with t-butyl hydroperoxide (tBH) induced thiol oxidation in thioredoxin-1. After exposure of cells to tBH, cells were lysed in the presence of alkylating agents. Following reduction with TCEP, free thiols were labeled with 10 kDa PEG conjugated to maleimide. Thus, in this technique, each oxidized thiol increases by 10 kDa the apparent molecular weight of a protein of interest, as analyzed by western blotting. In the case of trx-1, which contains intramolecular disulfides, each disulfide reduced yields 2 thiols and therefore increases the apparent MW by 20 kDa. FIG. 10C shows fluorescence microphotographs of cells using the same conditions as in FIG. 10B. Thiol oxidation was detected histochemic ally in fixed cells. After staining the cells for both oxidized thiols and trx-1, direct FRET was detected between CPM and the Alexa488-labeled trx-1 antibody by laser scanning confocal microscopy.

FIG. 11 shows fluorescence microphotographs comparing direct FRET and acceptor photobleaching. Cells were double labeled for trx-1 (acceptor, pseudo-colored red) and oxidized thiols (—S—S—, donor, pseudo-colored green) at baseline (top row) and after treatment with t-butyl hydroperoxide (tBH) (bottom row). The direct FRET image shows the fluorescence resonance energy transfer from the pre-bleach —S—S— signal to the pre-bleach trx-1 channel. Note the marked increase in direct FRET after treatment tBH. Arrows demarcate the area of photobleaching of the acceptor (red) and the corresponding regions of increased fluorescence of the donor (green). Note that that there is a much larger increase in donor fluorescence after tBH treatment, despite the fact that baseline trx-1 fluorescence and the level of bleaching were lower in the tBH-treated cells; this is mirrored in the increased FRET efficiency [1-(pre-bleach F/post-bleach F)]. Thus, direct FRET and acceptor photobleaching yield identical results.

FIG. 12 shows a negative control for FRET with beta-synuclein, which does not possess any cysteine residues in its primary sequence. As expected, no FRET was detected between oxidized thiols and Alexa488-labeled beta-synuclein antibody. Western blot analysis confirmed the expression of endogenous beta-synuclein in this cell line.

FIG. 13 is a schematic showing the detection of oxidized cysteines to disulfides. Together with the reaction (green line), a positive control (red line) and a negative one (yellow line) are performed. The level of fluorescence in the analyzed samples will range between the higher possible signal (positive control) and the background signal of the negative control (see inset).

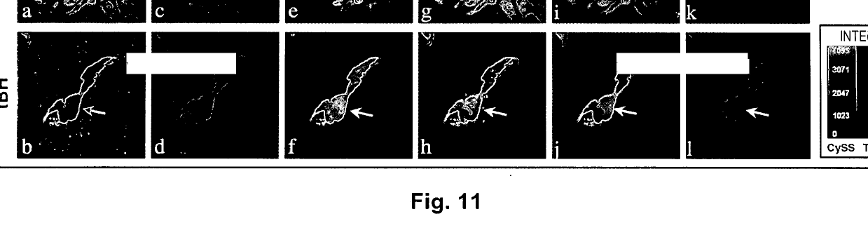

FIG. 14 shows histochemical labeling of oxidized thiols in cells exposed to t-butyl hydroperoxide (tBH), combined with immunofluorescence detection of thioredoxin-1. tBH induces a dramatic increase of thiol labeling. As expected, no labeling occurs in the control reaction, where TBP is omitted.

FIG. 15 shows how FRET reveals oxidized thiols in Trx-1. The schematic depicts FRET between two fluorophores labeled on the same protein. Cells were thiol-labeled as described above and were also immunolabeled for Trx-1, using AlexaFluor 488-conjugated primary antibody. Cells were then excited at 405 nM, which normally would not cause fluorescence in the green range (520 nM). Without the reducing step, there is no FRET (labeled “Control Reaction”). With the reducing step but without tBH, some amount of FRET is observed (for TBP ‘+’ and tBH ‘−’). Treatment with tBH results in a marked increase in FRET, indicating an increase in oxidized thiols in Trx-1.

FIG. 16 shows that there is no thiol oxidation in beta-synuclein, which contains no cysteine residues. Western blotting and direct immunofluorescence show abundant beta-synuclein in the cells, and there is abundant thiol labeling of tBH-treated cells (see FIG. 21), but there is no thiol labeling or FRET of beta synuclein. Similar results have been obtained in vivo with alpha-synuclein.

FIG. 17 is a schematic showing the labeling of sulfenic acid and free thiols with NBD-Cl. On the right side of the panel, the graph depicts the expected results from the reaction, the positive and the negative control.

FIG. 18 is a schematic showing the detection of nitrosocysteines. The procedure is similar to the cysteine labeling on (in FIG. 10), with some modification. In particular, ascorbate is added to reduce nitrosothiols to thiols. Together with the reaction (green line), a positive control (red line) and a negative one (yellow line) are performed.

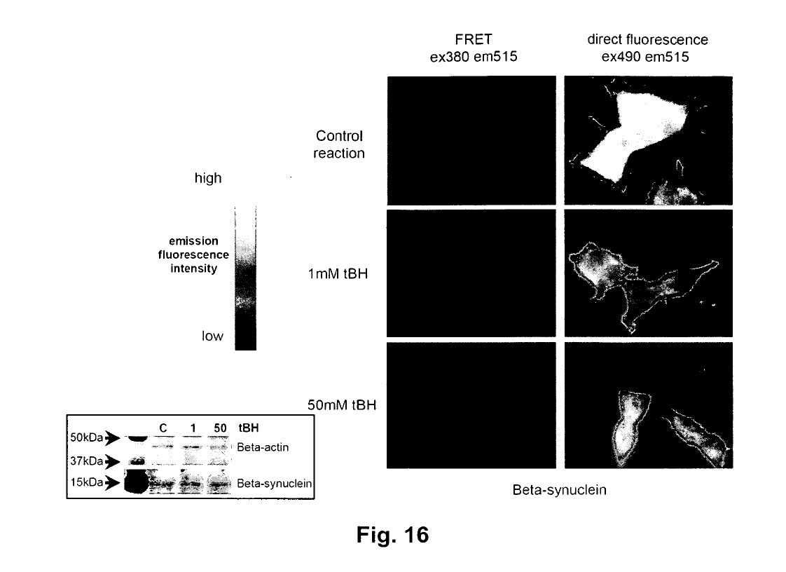

FIG. 19 shows a direct method for fluorescent labeling of protein carbonyls. FIGS. 19A-19B shows the comparison of the structures of DNPH and the fluorescent hydrazine compound. FIGS. 19C-19D show human neuroblastoma cells stained for carbonyls (green) and glucose transporter 1 (red). FIG. 19C is a fluorescence microphotograph of control cells treated with vehicle. FIG. 19D is a fluorescence microphotograph of cells treated with 50 nM rotenone, where a large increase in carbonyls is observed after treatment with rotenone.

FIG. 20A is a schematic showing the overview of the two convergent approaches used to identify mitochondrial proteins containing oxidized thiols following chronic exposure to rotenone. A control reaction was performed omitting the reducing step (asterisk). In this reaction, no free thiols should be present in the sample and, therefore, no proteins should be purified with either approach.

FIGS. 20B-20C are Coomassie stains of the eluted mitochondrial proteins from the thiol sepharose affinity chromatography. FIG. 20B shows a striking increase of oxidized proteins is evident in the substantia nigra of rotenone animals, as revealed by the number and the intensity of the bands (compare lanes 3 and 5). No major differences are visible in the striata (compare lanes 2 and 4). As expected, there are no eluted proteins in the control reaction (lane 6). FIG. 20C shows the loading control, where the flow through was resolved through SDS-PAGE and stained with Coomassie Blue. SN=ventral midbrain; ST=striatum.

FIG. 21 shows a redox western blot of thioredoxin (Trx-1) after tert-butylhydroperoxide (tBH), a strong oxidant. Because of PEG labeling, the molecular weight of trx-1 increases by 10 kDa for each oxidized thiol. This technique provides a semiquantitative assessment of oxidation status of candidate proteins of interest.

FIG. 22 shows fluorescence microphotographs of oxidized thiol staining and tyrosine hydroxylase immunofluorescence in substantia nigra of a control animal (top) and a rotenone-treated animal (bottom). Note the marked increase in oxidized thiols after rotenone and that virtually all TH-positive neurons are oxidized. Images were obtained by laser scanning confocal microscopy (LSCM).

FIG. 23 shows fluorescence microphotographs of combined imaging of GFAP (red), carbonyls (green), and tyrosine hydroxylase (blue) in substantia nigra from a control animal (top) and a rotenone-treated animal (bottom). Note the marked increase in carbonyls after rotenone and the fact that much of the increase is in the neuropil, apparently associated with astrocytic processes. Images were obtained by laser scanning confocal microscopy (LSCM).

FIG. 24A shows fluorescence microphotographs of dopaminergic neurons correlating presence of tyrosine hydroxylase and oxidized thiols. Bottom panels show cells treated with rotenone (labeled “rotenone”), where top panels show cells without treatment (labeled “control”). The left panels show staining of tyrosine hydroxylase (green), the middle panels show labeling of oxidized thiols (blue), and the right panels show the merged composite of the microphotographs shown in the left and middle panels. White arrows show cells that lack tyrosine hydroxylase but contain oxidized thiols.

FIG. 24B shows fluorescence microphotographs of dopaminergic neurons correlating presence of transferrin and oxidized thiols. Bottom panels show cells treated with rotenone (labeled “rotenone”), where top panels show cells without treatment (labeled “control”). The left panels show staining of transferrin (green), the middle panels show labeling of oxidized thiols (blue), and the right panels show FRET detection (red).

FIG. 24C shows Western blots of substantia nigra from a control animal (lanes labeled “C”) and a rotenone-treated animal (lanes labeled “R”). The four lanes on the left were from samples treated with dithiothreitol (“DTT”), where the four lanes on the right were not treated with DTT. Black arrow shows reduced monomeric transferrin, black arrow head shows oxidized transferrin engaged in an intramolecular disulfide bond.

FIG. 25 shows fluorescence microphotographs of a tumor that has been treated with ethyl pyruvate (“ep”) and gentamicin (“gen”). The top microphotograph shows oxidized thiols (red) and nuclei (green). The bottom microphotograph shows the intensity of oxidized thiols, where white indicates high intensity and blue indicates low intensity as indicated on the color scale.

FIG. 26 shows fluorescence microphotographs of tumor tissues treated with various agents. The intensity shows the level of oxidized thiols, where white indicates high intensity and blue indicates low intensity on the color scale. Skin tissue is shown (image within box labeled “skin”). Tumor tissues are shown for treatment with Ringer's solution (images within box labeled “Ringer”), for treatment with gentamycin (labeled “gen”), for treatment with oxaliplatin (labeled “ox”), for treatment with ethyl pyruvate (labeled “ep”), and for melphalan (labeled “mel”).

FIG. 27 shows fluorescence microphotographs of tumor tissues treated with a combination of agents. The intensity shows the level of oxidized thiols, where white indicates high intensity and blue indicates low intensity on the color scale. Tumor tissues are shown for control (image within box labeled “skin”), for treatment with Ringer's solution (images within box labeled “Ringer”), for treatment with oxaliplatin and ethyl pyruvate (labeled “ox+ep”), for treatment with melphalan and ethyl pyruvate (labeled “mel+ep”), and for treatment with gentamicin and ethyl pyruvate (labeled “gen+ep”).

FIG. 28 shows fluorescence microphotographs of tumor tissues treated with a oxaliplatin (top two panels labeled “ox”) or ethyl pyruvate (bottom two panels labeled “ep”). The left panel shows stained nuclei (green), the middle panel shows oxidized thiols (red), and the right panel shows composite images of the microphotographs shown in the left and middle panels (overlap indicated in yellow).

FIG. 29 shows fluorescence microphotographs of tumor tissues treated with Ringer's solution (top two panels labeled “ringer”) or mel (bottom two panels labeled “mel”). The left panel shows stained nuclei (green), the middle panel shows oxidized thiols (red), and the right panel shows composite images of the microphotographs shown in the left and middle panels (overlap indicated in yellow).

FIG. 30 shows fluorescence microphotographs of tumor tissues treated with gentamycin (top two panels labeled “gen”) or a combination of ethyl pyruvate and mel (bottom two panels labeled “ep+mel”). The left panel shows stained nuclei (green), the middle panel shows oxidized thiols (red), and the right panel shows composite images of the microphotographs shown in the left and middle panels (overlap indicated in yellow).

FIG. 31 shows fluorescence microphotographs of skin tissue (top panel labeled “skin”), of tumor tissue treated with oxaliplatin and ethyl pyruvate (middle two panels labeled “ox+ep”), and of tumor tissue treated with gentamycin and ethyl pyruvate (bottom panel labeled “gen+ep”). The left panel shows stained nuclei (green), the middle panel shows oxidized thiols (red), and the right panel shows composite images of the microphotographs shown in the left and middle panels (overlap indicated in yellow).

FIG. 32A shows infrared scanner images of liver tissue samples (labeled from 1 to 10) treated with various agents. On the left, original images of tissues are shown. On the right, images are shown with region of interest (“ROI”) outlined in blue and with integrated intensity obtained within the ROI.

FIG. 32B is a graph showing levels of oxidized thiols in samples that were analyzed using infrared scanner images. The table shows the sample number, the slide name, and levels of oxidized thiols determined from standardized integrated intensity within the ROI.

FIG. 33A shows infrared scanner images of tissue samples incubated in redox buffer at a redox potential (“Eh”) from −300 mV to −150 mV. On the left, original images of tissues are shown. On the right, images are shown with region of interest (“ROI”) outlined in white and with integrated intensity obtained within the ROI.

FIG. 33B is a graph showing the disulfides redox standard curve derived from liver tissue imaged using an infrared scanner. The presence of disulfides was determined from Alexa680 emission

FIG. 33C is a graph showing the disulfides redox standard curve derived from the SH-SY5Y human neuroblastoma cells imaged using fluorescence microscopy. The presence of disulfides was determined from CPM/SYTOX emission.

FIG. 33D shows infrared scanner images of different tissue samples treated with various agents (labeled from #1 to #17).

FIG. 33E shows infrared scanner images of different tissue samples treated with various reagents, where the region of interest (“ROI”) outlined in blue and integrated intensity obtained within the ROI are shown.

FIG. 33F is a graph showing levels of disulfides in samples that were analyzed using infrared scanner images. The table shows the sample ID and levels of disulfides.

### DETAILED DESCRIPTION

Methods of detecting concentrations of disulfides and the oxidation states of cells are described herein. Such methods are also compatible with standard histochemical and immunohistochemical staining procedures, thus allowing multiple stains to be used in conjunction with the detection of the cell's redox state. For example, by monitoring the fluorescence resonance energy transfer (FRET) between a fluorochrome-labeled specific primary antibody to a cell protein of interest and a fluorochrome-labeled thiol probe, the thiol oxidation state can be detected in the cell of interest. Such methods have successfully been used to demonstrate that thiol oxidation occurs selectively in the dopaminergic neurons of the substantia nigra in brains of animals having experimental Parkinson's disease, which are the same neurons that are lost selectively in the disease. The disclosed methods allow detection of oxidative imbalance pinpointed to particular cells and proteins, and thus find use in diagnostics and treatment for disease.

The use of numerical values in the various ranges specified in this application, unless expressly indicated otherwise, are stated as approximations as though the minimum and maximum values within the stated ranges are both preceded by the word “about.” In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases. All references are fully incorporated by such reference herein, solely to the extent of their technical disclosure and only such that it is consistent with this disclosure.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

As used herein, the term “subject” refers to members of the animal kingdom including but not limited to human beings that are treated using the methods and compositions described herein.

“Treatment” of a medical condition associated with a fibrotic response or proliferation or treatment or preventing a fibrotic response or proliferation or scarring means administration to a subject by any suitable route and dosage regimen of a drug product comprising an active agent with the object of ameliorating (e.g., attenuating, alleviating, reducing and/or normalizing) any symptom and/or indicia associated with the medical condition, including, without limitation, any testable parameter, whether or not subjective, such as, without limitation, pain levels, or objective, such as, without limitation, levels of biomarkers in blood sample of a subject, or lesion size. Likewise “treating” such a medical condition may result in amelioration of any symptom and/or indicia associated with the medical condition in a subject.

As used herein, “pharmaceutically-acceptable,” means acceptable for use in humans and animals. “Excipients” include, without limitation, one or more suitable: vehicle(s), solvent(s), diluent(s), pH modifier(s), buffer(s), salt(s), colorant(s), rheology modifier(s), lubricant(s), filler(s), antifoaming agent(s), erodeable polymer(s), hydrogel(s), surfactant(s), emulsifier(s), adjuvant(s), preservative(s), phospholipid(s), fatty acid(s), mono-, di- and tri-glyceride(s) and derivatives thereof, wax(es), oil(s) and water. The choice of excipient depends on the dosage form in question. The drug product may be administered, without limitation, intravenously, intramuscularly, orally, topically, intratumorally, intraperitoneally, intrathecally, rectally, vaginally, nasally, optically, buccally, transdermally, subdermally, intradermally, etc., as is appropriate and/or desirable for treatment. Parenteral administration may require at a minimum buffers and salts to match physiological conditions, and thus includes salt and buffer, such as, without limitation, normal saline or phosphate-buffered saline. Depending on the solubility of the compound (active ingredient), the dosage form may be aqueous, micellular (including liposomes) or lipophilic. Formulation of a drug product and choice of suitable excipient(s) with adequate bioavailability is within the average skill of those in the pharmaceutical and formulary arts. The compound may be administered via any useful delivery route, including, without limitation, topically, orally or parenterally, and the drug product/dosage form is tailored to the desired delivery route. For example and without limitation, an HCl salt of a compound described herein may be administered topically, intravenously or intramuscularly in normal saline, or may be administered in tablet or capsule form with appropriate excipients. A large variety of dosage forms are known in the pharmaceutical arts, and many of which may be appropriate for treatment using the methods and compositions described herein (see generally, Troy, D B, Editor, Remington: The Science and Practice of Pharmacy, 21st Edition, Lippincott, Williams & Wilkins (2005)).

Herein described are methods of labeling carbonyls in histological samples, for example. Such methods allow for discrimination of the specific subcellular populations most affected by oxidation in complex tissues such as brain. Such methods also allow for the sub-cellular compartments where oxidation is targeted to be identified. In certain embodiments it is possible to determine if a certain protein is oxidized as well as its spatial relationship to other cells and tissues.

The methods described herein comprise labeling oxidized groups on a protein with an oxidation labeling reagent comprising a first member of a FRET pair, and detecting the target protein by labeling the protein with a binding reagent specific for the protein wherein the binding reagent comprises a second member of the FRET pair. As used herein the term “oxidation labeling reagent” is intended to mean a reagent that specifically binds chemical moieties that are capable of (or have been) oxidized such as, for example, carbonyl groups. The term “FRET” is an acronym for fluorescence (or Forster) resonance energy transfer. A “FRET pair” includes two members—a donor compound and an acceptor compound—in which FRET can occur when the members of the FRET pair are in sufficiently close proximity to each other, which is typically, but not exclusively from about 10 Å (10 Angstrom) to about 100 Å. In theory (without any intention to be bound by this theory), it occurs primarily because the acceptor dipole interacts or resonates with the donor dipole. The conditions for this mechanism are that the fluorescent emission spectrum of the energy donor overlaps the absorption spectrum of the energy acceptor. Also, it is believed (without any intention to be bound by this theory) that donor and acceptor transition dipole orientations must be approximately parallel. Two non-limiting examples of FRET pairs, as described herein are CPM/Alexa488 and CPM/FITC. Notably, such techniques can be adapted to other probes with different spectral properties, excitable, for instances, in the far-red field. This strategy can reduce or eliminate the background noise due to UV-excitable molecules that are naturally present in the cell (i.e. tyrosine residues).

A large variety of fluorophores is available and can find use in the methods described herein, for example and without limitation: Alexa Fluors (Molecular Probes/Invitrogen) and Dylight Fluors (Thermo Fisher Scientific). These fluorophores have an emission spectra that span a wide range, including ultraviolet, near-ultraviolet, visible, near-infrared, and infrared ranges. As such, the oxidation state of the target proteins can be identified and/or quantified by any useful fluorescence method, such as by fluorescent microscopy, fluorescent plate readers, infrared scanner analysis, spectrophotometers, fluorescent-activated cell sorters (FACS), and fluorescent scanners (e.g., gel/membrane scanners).

Although samples can be analyzed with a laser scanning confocal microscope or a microplate spectrofluorometer, the method can be easily adapted to other devices (i.e. FACS cell sorter) for applications in other fields. FRET can be detected directly or indirectly. Direct detection of FRET is performed exciting the donor (CPM) and detecting the signal emitted by the acceptor (FITC or Alexa488). As used herein the term “signal” means any detectable event (whether direct or indirect) indicative of FRET, and includes without limitation, emission of a photon. FRET is detected indirectly using the method described by Karpova and co-workers (T. S. Karpova et al., J Microsc 209, 56-70, 2003). Briefly, after having acquired the signal for both fluorophores, the acceptor (CPM) is destroyed (bleached, in a specific region of interest (ROI), through high-intensity laser stimulation. After bleaching, the signals of both donor and acceptor are measured again; if FRET is detected, the intensity of the donor will increase after the bleaching process. Detection of FRET indicates that the fluorophores on the oxidation labeling reagent and the binding reagent are in close proximity (about 50 Å away), therefore on the same protein. FRET efficiency (F_{eff}) can be calculated by confocal software (according to the equation F_{eff}=1−(emₚᵣₑ/emₚₒₛₜ), where F_{eff}=FRET efficiency, emₚᵣₑ=donor emission prebleaching, emₚₒₛₜ=donor emission post bleaching.

The term “binding reagent,” includes any compound, composition or molecule capable of specifically or substantially specifically (that is with limited cross-reactivity) binding another compound or molecule, which, in the case of immune-recognition contains an epitope. In many instances, the binding reagents are antibodies, such as polyclonal or monoclonal antibodies. “Binding reagents” also include derivatives or analogs of antibodies, including without limitation: Fv fragments; single chain Fv (scFv) fragments; Fab′ fragments; F(ab′)2 fragments; humanized antibodies and antibody fragments; camelized antibodies and antibody fragments; and multivalent versions of the foregoing. Multivalent binding reagents also may be used, as appropriate, including without limitation: monospecific or bispecific antibodies, such as disulfide stabilized Fv fragments, scFv tandems ((scFv)fragments), diabodies, tribodies or tetrabodies, which typically are covalently linked or otherwise stabilized (i.e., leucine zipper or helix stabilized) scFv fragments. “Binding reagents” also include aptamers, as are described in the art.

Methods of making antigen-specific or non-specific binding reagent compositions, including antibodies and their derivatives and analogs, are well-known in the art. Polyclonal antibodies can be generated by immunization of an animal and recovery of plasma. Monoclonal antibodies can be prepared according to standard (hybridoma) methodology. Antibody derivatives and analogs, including humanized antibodies can be prepared recombinantly by isolating a DNA fragment from DNA encoding a monoclonal antibody and subcloning the appropriate V regions into an appropriate expression vector according to standard methods. Phage display and aptamer production methods are well known and permit in vitro clonal amplification of antigen-specific binding reagents with high affinity and low cross-reactivity. The combination of a binding reagent and the protein, compound, molecule, epitope, ligand, antigen, etc. to which the binding reagent binds is a binding pair. The binding reagent and the protein, compound, molecule, epitope, ligand, antigen, etc. to which the binding reagent binds individually are binding partners of the binding pair.

The term “dye” includes any compound, composition or molecule capable of emitting light. In many instances, the dyes are fluorophores, which emit light in the visible region of light. In other instances, the dyes can emit light in the non-visible regions of light, such as ultraviolet, near-ultraviolet, near-infrared, and infrared. For example and without limitation, examples of dyes include: quantum dots; nanoparticles; fluorescent proteins, such as green fluorescent protein and yellow fluorescent protein; heme-based proteins or derivatives thereof; carbocyanine-based dyes, such as IRDye 800CW, Cy 3, and Cy 5; coumarin-based dyes, such as (7-diethylamino-3-(4′-maleimidylphenyl)-4-methylcoumarin) (CPM); fluorine-based dyes, such as fluorescein, fluorescein isothiocyanate (FITC); and numerous Alexa Fluor® dyes and Alexa Fluor® bioconjugates, which absorb in the visible and near-infrared spectra. The emission from the dyes can be detected by any number of methods, including but not limited to, fluorescence spectroscopy, fluorescence microscopy, fluorescent plate readers, infrared scanner analysis, laser scanning confocal microscopy, laser spectrophotometers, fluorescent-activated cell sorters (FACS), and fluorescent scanners (e.g., gel/membrane scanners).

A biological sample is any sample obtained from a biological source, including without limitation: cells, cell extracts, biological fluids, tissues, organs or other systems. Biological samples include, without limitation: cell or tissue homogenates, microscope slides (thin layers), and cell cultures. These samples can also be obtained from patients, specimens, or animals with different disease states. These disease states include, without limitation: oxidative stress and cancer.

In one embodiment, methods are provided for identifying the oxidative state of a protein in a biological sample. Similarly, methods of labeling oxidized thiol or carbonyl groups in a biological sample are provided. The method comprises: alkylating or otherwise blocking free thiols in the biological sample with a blocking agent; reducing disulfide bonds (oxidized thiols) in the biological sample with a reducing agent; and labeling remaining free thiols with a first member of a FRET pair attached to an alkylating group. The method also may comprise binding a binding reagent to a target protein in the biological sample, wherein the binding reagent is attached to a second member of the FRET pair. By “attached” it is intended to mean that constituents are covalently linked (e.g., conjugated) or bound together non-covalently with sufficient strength/affinity such that the methods described herein can be performed. A method of detecting oxidized thiol groups in a biological sample includes detecting FRET in a sample prepared by a method of labeling oxidized thiols in a biological sample as described herein, wherein such labeling is indicative of the presence of oxidized thiol groups co-localized in the biological sample with the target protein.

The blocking agent(s) described herein can be an alkylating agent, such as, without limitation: haloalkyl compounds, such as haloalkanes (e.g., monobromobimane) and alpha-halocheto compounds (e.g., iodoacetic acid, iodoacetamide); N-alkylmaleimide (e.g., N-ethylmaleimide); alkylmethanethiosulfonate (e.g., methyl methanethiosulfonate); p-hydroxymercurybenzoate (e.g., mercury orange from Molecular Probes, which is fluorescent per se); Ellman's reagent (5,5′-dithiobis(2-nitrobenzoic acid) and its derivatives, metal ions (e.g., zinc, mercury, gold and silver. Certain of the alkylating reagents described above may fluoresce per se. Nevertheless, any of the described compounds can be attached to a fluorophore, typically by a linking group, in order to produce a member of a FRET pair attached to an alkylating group useful in the methods described herein.

The reducing agent may be any useful reducing agent for breaking disulfide bonds in a protein sample that can be effective in the methods described herein. Non-limiting examples of reducing agents include: Cleland's reagent, DTT (dithiothreitol) or DTE (dithioerythriol); 2-mercaptoethanol (β-mercaptoethanol); 2-mercaptoethylamine; and trivalent phosphines, such as TBP (tributylphosphine) or TCEP (tris(2-carboxyethyl)phosphine).

In one embodiment, the methods described herein obviate the need for a washing step or any step that exposes the biological sample to oxidizing conditions, such as, without limitation, prolonged exposure to air, after exposing the biological sample to the reducing agent. Exposure of the biological sample to oxidizing conditions can result in spontaneous re-oxidation of the reduced sulfhydryl groups and may lead to spurious results.

In another embodiment, a method of labeling carbonyl groups in a sample is provided. The method comprises conjugating protein in a sample to an oxidation labeling reagent, the labeling reagent comprising a first member of a fluorescence resonance energy transfer (FRET) pair and a carbonyl-reactive group. The method also may comprise binding a binding reagent to a target protein in the biological sample, wherein the binding reagent is attached to a second member of the FRET pair. A method of detecting carbonyl groups in a biological sample includes detecting FRET in a biological sample prepared by a method of labeling carbonyl groups in a biological sample as described herein, as indicative of the presence of oxidized thiol groups co-localized in the biological sample with the target protein. In one embodiment, the carbonyl-reactive group is a hydrazine. In one embodiment, the first member of the FRET pair is attached to the hydrazine, or a hydrazine derivative, such as 3,5-dinitro-phenylhydrazine, by any useful means, including for instance by a linker.

In another embodiment, methods of determining if an agent (e.g., a compound, composition, drug, molecule, etc.) can affect the cellular oxidation state in a tumor cell and/or determining if an agent can prevent tumor cell growth are provided. As described herein, the oxidation state within in a cell can be correlated with the effectiveness of a chemotherapeutic agent. For example, an effective chemotherapeutic agent would be more likely to induce oxidation of thiols within the tumor cells and, therefore, more likely to prevent tumor growth. A wide range of chemotherapeutic agents can be used alone or in combination. For example and without limitation, these agents include platinum-based drugs, antibiotics, antioxidants, alkylating agents, antimetabolites, cytokines, and free radical scavengers.

The methods comprise: contacting one or more tumor cells with one or more chemotherapeutic agents; detecting and/or quantifying the presence of oxidized thiols or carbonyls in the tumor cell, where a control is also obtained by detecting and/or quantifying the presence of oxidized thiols or carbonyls in tumor cells that are not contacted with the chemotherapeutic agent(s); and comparing the oxidation level of the cells treated with the chemotherapeutic agent(s) to the control. The presence of oxidized thiols or carbonyls in the tumor cell (and the control) can be determined by several methods. For example and without limitation, methods include labeling only the oxidized thiols by labeling only the carbonyl, or by labeling the oxidized thiols and labeling another protein or carbonyl group within the tumor cell, detectable by such means as by FRET. Detection and/or quantification of the presence of oxidized thiols or carbonyls may be by fluorescent imaging, confocal microscopy, infrared scanning analysis, spectrophotometry, and/or fluorescent-activated cell sorting.

Although the methods disclosed herein are useful for any compatible protein disclosed herein or know in the art, certain exemplary embodiments are contemplated and include, but are not limited to: proteins involved in signaling through phosphorylation such as Ask (Cross, J V et al. (2006) Antiox. Redox Signal. 8, 1819-27), PP2a (Cho, S H et al. (2004) FEBS Lett. 560, 7-13), and PTEN (Shaulian, E et al. (2002) Nature Cell. Biol. 4, E131-6); proteins involved in the cell cycle such cdc25 (Rudolph J (2005) Antiox. Redox Signal. 7, 761-7); proteins involved in transcription such as ΛP-1 (Shaulian, E et al. (2002) Nature Cell. Biol. 4, E131-6) and NF-kB (Cross, J V et al. (2006) Antiox. Redox Signal. 8, 1819-27); proteins regulating ionic fluxes such as NMDAR (Rudolph J (2005) Antiox. Redox Signal. 7, 761-7) and RyR (Hamilton S L, et al. (2000) Antiox. Redox Signal. 2, 41-5); proteins involved in mitochondrial function such as proteins of respiratory complex I (Hurd T R, et al. (2008) J. Biol. Chem. 283, 24801-15); proteins of the permeability transition pore (Costantini P. et al. (1996) J. Biol. Chem. 22, 6746-51); proteins involved in cancer such as HMGB1 (Shau D, et al. (2008) FEBS Lett. 582, 3973-8) and glutathione-S transferase; proteins involved in redox regulation such as thioredoxins (Watson W H, et al. (2003) J Biol Chem. 278, 33408-15) and glutaredoxins (Fernandes A P, et al. (2004) Antioxid Redox Signal. 6, 63-74); and proteins involved in neurodegenerative diseases (Mastroberardino P G, et al. (2009) Neurobiol Dis., 14, and Cho S H, et al. (2009) Science 324, 102-5). Further included are the carbonylated proteins presented in Ellis E M, Pharmacol Ther. 2007 July; 115(1):13-24, which is incorporated by reference in its entirety.

In certain other embodiments, methods of identifying pathology such as a disease or exposure to a toxic compound in a subject, such as a human or animal is provided. The method comprises obtaining a biological sample from a subject and determining the oxidation state in the biological sample by any of the methods described herein. The results of such an assay can provide an indication of pathology.

In another embodiment, a method of screening compounds to determine if such a compound can restore a cell from abnormal cellular oxidation state is provided. The method comprises: contacting one or more cells having an abnormal cellular oxidation state with a compound of interest; and detecting and/or quantifying in the cell the presence of oxidized thiol or carbonyl groups as compared to a control which is not contacted with the compound. An abnormal oxidation state may be caused by pathology such as disease, cancer, genetic defects and/or a toxin. Such toxins may be a pesticide, such as one or rotenone and paraquat. Moreover, the compound tested may be an antioxidant.

In another embodiment, a method of determining if a compound (e.g., a drug, a molecule, etc.) can affect the cellular oxidation state in a cell, such as a cancer cell is provided. Such methods comprise: contacting one or more cancer cells with one or more compounds such as chemotherapeutic agents; detecting and/or quantifying the presence of oxidized thiols or carbonyls in the cancer cell; and comparing the oxidation level of the cells treated with the chemotherapeutic agent(s) to a control, wherein increased oxidation level in the tumor cells treated with the chemotherapeutic agent(s) is indicative of the ability of the agent(s) to induce oxidation of thiols and thereby preventing tumor growth. Thus, in a related embodiment, a method of determining if an agent can prevent tumor growth is provided. Cells can be obtained from any organ or tissue, such as, skin, liver, or spleen. The chemotherapeutic agent tested may be a platinum-based chemotherapy drug, an antibiotic, an antioxidant, alkylating agent, anti-metabolite, cytokine, and/or a free radical scavenger.

The tumor cells can be obtained from any organ or tissue, such as, skin, liver, or spleen. The chemotherapeutic agent tested may be a platinum-based chemotherapy drug, an antibiotic, an antioxidant, alkylating agent, antimetabolite, cytokine, and/or a free radical scavenger.

In yet another embodiment, a kit is provided. A kit can comprise packaging, as is commercially acceptable and contains, for example, a first container having an alkylating agent, a second container having a reducing agent and a third container having an oxidation detection reagent comprising a first member of a FRET pair attached to an alkylating group. The kit may further comprise a fourth container having a binding reagent comprising a second member of the FRET pair. As automated sample processing systems are becoming more prevalent (e.g., instruments for performing automated assays, for example, as are described in a large number of published patent documents), the kit may further comprise a cartridge suitable for use in such an automated sample processing system, such that the first, second, third, and optionally fourth containers are elements configured within a cartridge for use in an automated sample processing system. For example, the kit may be compatible with automated systems already available including the DAKO AUTOSTAINER™, which allows a multitude of staining procedures and processing of specimens to be performed automatically, rapidly and reproducibly. As such it is well-suited for immunostaining of tissue sections, cytospins and cell smears. The alkylating agent, reducing agent, first member of a FRET pair, and binding reagent include, without limitation, all examples of such reagents provided herein.

In another embodiment, the kit comprises packaging, as is commercially acceptable and comprises a first container having an oxidation labeling reagent, the labeling reagent comprising a carbonyl-reactive group and a first member of a fluorescence resonance energy transfer (FRET). The kit may further comprise a second container comprising a binding reagent having a second member of the FRET pair. The kit may comprise a cartridge suitable for use in an automated sample processing system, such that the first and optionally second containers are elements configured within a cartridge for use in an automated sample processing system, such as the DAKO AUTOSTAINER™. The labeling reagent and binding reagent conjugated to a second member of the FRET pair include, without limitation, all examples of such reagents provided herein.

The following Examples are provided for illustration and, while providing specific example of embodiments described herein, are not intended to be limiting.

### Examples

### Example 1

### Immunohistochemical Detection of Carbonyls

Formation of carbonyls on amino acid residues in proteins is one of the main consequences of oxidative stress. Modification of carbonyl groups can be pursued in acid environment with molecules containing the functional group hydrazine. This strategy does not provide accurate absolute information as it is not highly specific; in fact, it targets all aldehydes and ketones, including those in carbohydrates, which do not necessarily represent the product of oxidation. Nonetheless, hydrazine labeling has been proven very useful for relative measures, comparing different populations. In such circumstances, this approach provides important clues about the amount of oxidation as well as the identity of oxidized proteins.

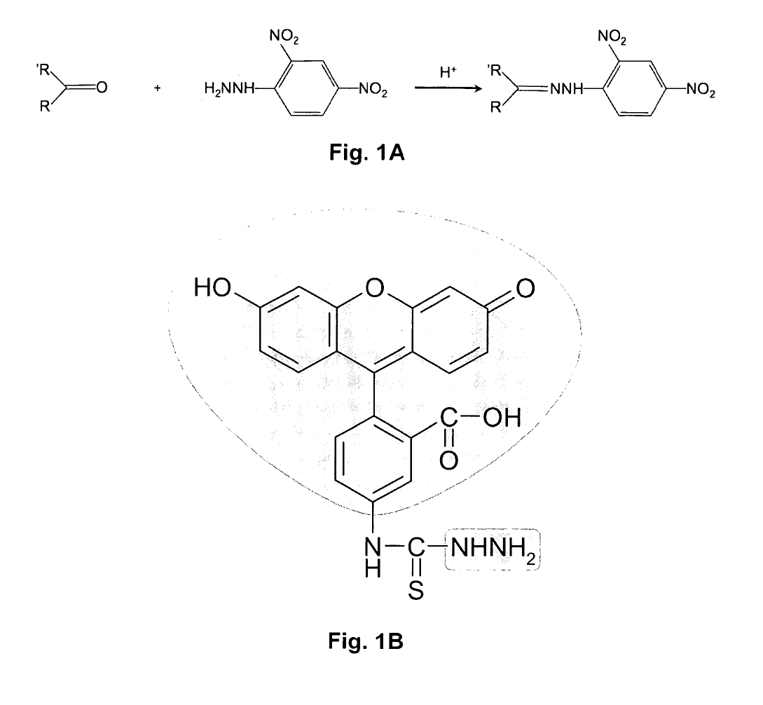

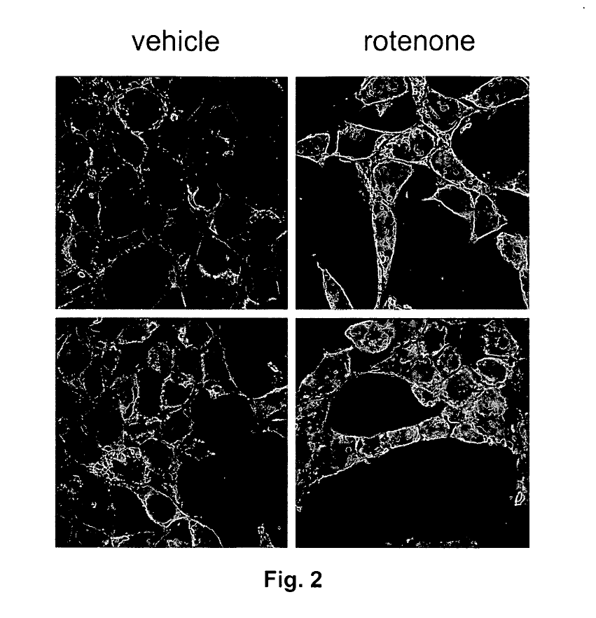

Technically, 3,5-dinitro-phenylhydrazine has been the most used reagent to label carbonyls. The principal advantage in the use of such a molecule relies in the wide number of commercial antibodies which successfully recognize the product of the reaction. To minimize non specific reactions and to make possible the FRET, a hydrazine which is directly conjugated to fluorescein is used, thus eliminating the steps involving the use of primary as well as secondary antibodies for DNPH (see, e.g., FIG. 1).

Cells are fixed in cold methanol for 10 minutes at −20° C. Paraformaldehyde and other keto group-containing (and therefore carbonyl group-containing) fixatives should be avoided. The method can be successfully applied to tissues as well.

Carbonyls are labeled with fluorescein-conjugated hydrazide (FITC-NN, FIG. 1, Molecular Probes, cat #F-121). F121 is prepared fresh in dimethyl-formamide. As the reaction between the hydrazine and the carbonyls occurs in acidic environment, the labeling is carried out in 50 mM sodium acetate (pH 4). FITC-NN is used 2 microM final; incubation is carried on 2 hours at room temperature.

Samples are washed in phosphate buffer solution (PBS) for 5 minutes; this step is repeated three times.

From this step onward, a classical immunohistochemistry staining can be performed to detect proteins of interest.

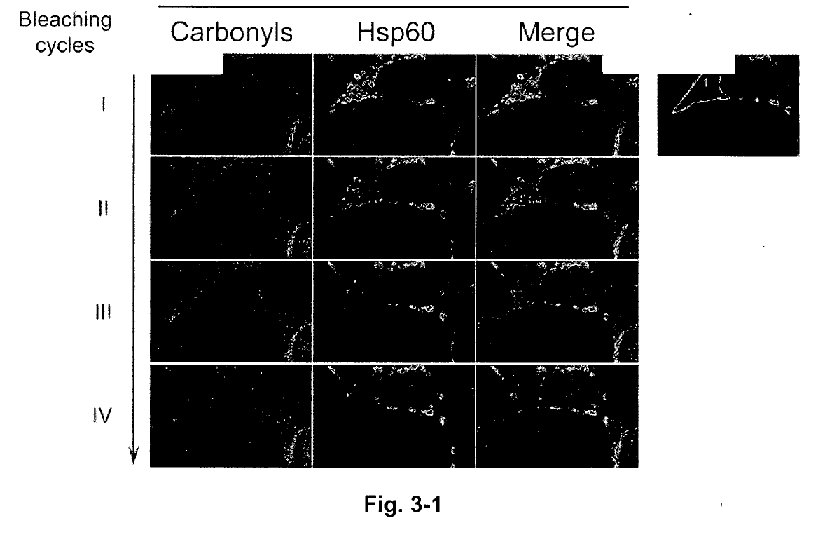

Images are acquired with a laser scanning confocal microscope. FIG. 2 shows fluorescence microphotographs obtained by laser scanning confocal microscopy, where labels are shown for carbonyls (green) and glyceraldehyde 3-phosphate dehydrogenase (red).

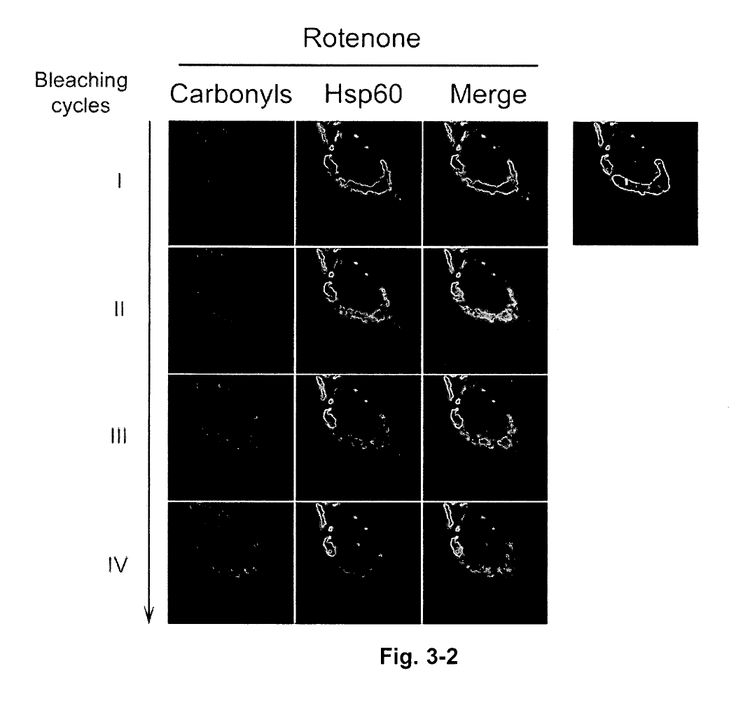

Anti-Hsp60 (Stressgen, used at 1:500) was conjugated with Cy3. The procedure can be carried out in a 96-well plate, which can be analyzed with a micro-plate spectrofluorometer. FRET between FITC-NN and a Cy3-labeled specific antibody can be studied to determine if the two fluorophores are in close proximity (<50 Å) and therefore if the protein of interest is oxidized. FIG. 3 shows the acceptor photobleaching method to determine whether the protein of interest is oxidized. First, the acceptor fluorophore is photobleached over four cycles. In FIG. 3, Cy3 was the acceptor fluorophore and the protein of interest was Hsp60, where Cy3 conjugated with anti-Hsp60 antibody. Second, the emission of the acceptor fluorophore and of the donor fluorophore are determined and compared. If a significant increase in the donor emission is observed after photobleaching the acceptor fluorophore, then the two fluorophores are determined to be in close proximity. FIG. 3 shows a significant increase in FITC emission after photobleaching of Cy3 (red) in rotenone-treated cells. Therefore, the two fluorophores are in close proximity and indicate that the protein of interest, here Hsp60, is oxidized.

### Example 2

### Histochemical Detection of Oxidized Thiols (Disulfides) in Proteins

All steps are carried out at room temperature (about 25° C.), unless indicated. The methods can be applied to cells as well as tissue samples. Cells are seeded and cultured on cover-slips. Cells are fixed in the following solution: 4% paraformaldehyde (fixative), 0.02% Triton X-100 (to permeabilize cells), 100 mM iodoacetamide, and 100 mM N-ethyl-maleimide (alkylating agents to block free thiols). The solution is maintained at pH 6.8. NOTE: (1) The pH is maintained at 6.8 through the entire process. This is crucial as prevents the formation of non-specific adducts of maleimide. The reaction occurs also at physiological pH (7.4) even if there is some risk of non-specific. (2) Adding the alkylating agents in the fixative solution prevents alteration of the redox status of the free thiols because of permeabilization of the membrane and exposure to atmospheric oxygen. (3) Using two alkylating agents in combination makes the blocking process more efficient.

Tissues are quickly dissected and (1) incubated 24 hrs in 4% paraformaldehyde, 100 mM iodoacetamide and 100 mM N-ethyl-maleimide (2) incubated in 48 hrs in 30% sucrose, 25 mM NaH₂PO₄, 10 mM K₂HPO₄. After this step, tissues can be preserved in standard cryoprotectant buffer (25% glycerin, 30% Ethylene Glycol, 50% 0.1M PO₄ at −20° C. NOTE: (1) For better preservation of the redox status of the tissue, the sucrose as well as the cryoprotectant solutions can be supplemented with 20 mM N-ethyl-maleimide. (2) Although this fixation procedure is highly recommended, the technique has been successfully applied to tissues that were conventionally fixed and processed in paraffin or OTC.

Specimens are washed 5 minutes in Phosphate Buffered Saline (PBS) to remove the excess of alkylating agents. This step is repeated three times.

Thiols in the form of disulfides are reduced incubating the samples 20 minutes in PBS containing 50 mM tris(2-carboxyethyl)phosphine hydrochloride (TCEP). Note: a control reaction is performed omitting TCEP. In this reaction, no free thiols are present; therefore, the entire signal that will be eventually detected is due to non-specific noise. This reaction can be used as a standard to set the background zero of the acquiring instrument.

The TCEP solution is removed and the labeling solution, 0.5 mM of CPM (7-diethylamino-3-(4′-maleimidylphenyl)-4-methylcoumarin) in PBS, is added. Samples are incubated for 30 minutes at room temperature. Note: (1) TCEP does not interfere with the functional group of the labeling molecule CPM, a maleimide. Therefore, further washing steps—which could result in spontaneous re-oxidation of the thiols—can be omitted. This is an important improvement compared to current approaches using DL-Dithiothreitol (DTT), which interferes with the alkylating reaction. (2) Maleimide—the functional group in the labeling compound used in this technique—is the most reactive group toward thiols (FIG. 2). The only current approach uses a less reactive compound, iodoacetamide (Y. Yang et al., Proc Natl Acad Sci USA 104, 10813-10817 (2007)). Importantly, this method avoids the use of biotinylated probes, which generate non-specific signal being biotin normally present in cells.

Samples are washed for 10 minutes in PBS. This step is repeated three times.

The following steps resemble a standard immuno-histochemistry procedure.

Samples are blocked with 5% goat serum in PBS, 30 minutes at room temperature.

Samples are incubated over night, at 4° C., with the primary antibody solution. Note: the primary antibody (e.g., thioredoxin-1 (R&D Systems, Minneapolis, Minn.) and transferrin (VAP-EN004, Stressgen, Victoria, BC, Canada)) can be custom-labeled with Alexa488 or FITC, which constitute a good FRET couple with CPM.

Samples are washed for 10 minutes in PBS. This step is repeated three times.

Sections or cover-slips are mounted on slides using an anti-fade medium.

Images are acquired using a laser scanning confocal microscope.

### Example 3

### Fluorescence Detection by Microplate Reader in 96-Well Plates

The two detailed protocols for the detection of oxidation by-products in histological samples are described above, where Example 1 describes the detection of carbonyls and Example 2 describes the detection of disufides. The procedures have been successfully applied for analyses with fluorescent microscopes as well as microplate readers.

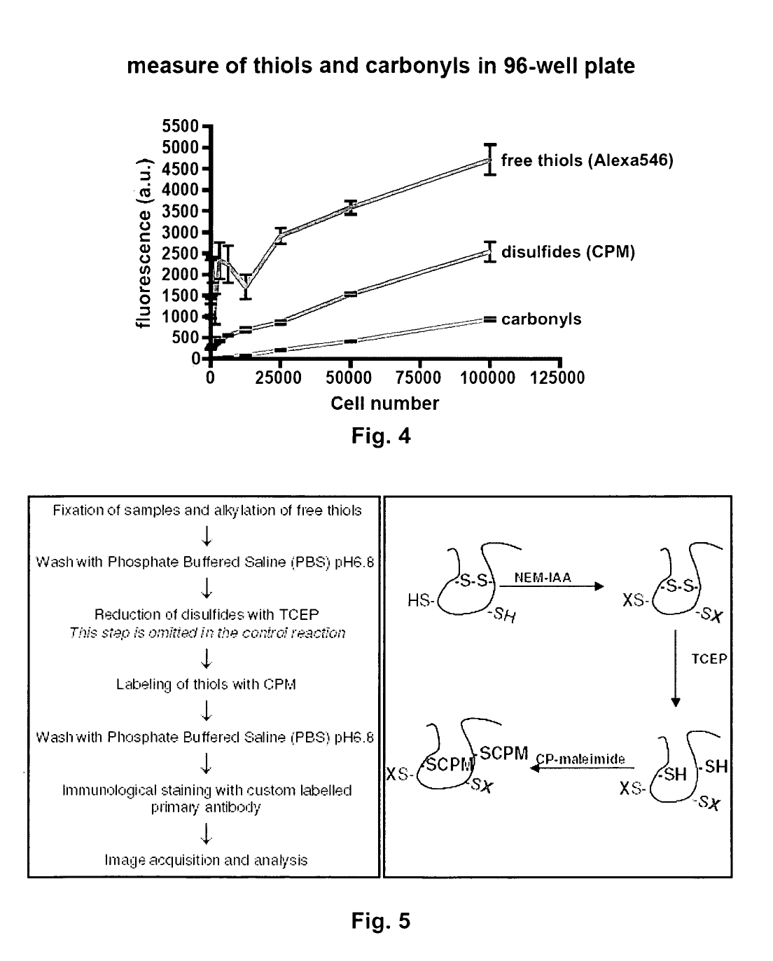

When the staining was carried out on 96-well plates, a linear response with cell densities between 20,000 and 75,000 cells per well was observed (FIG. 4). For experimental purposes, if the staining is performed on adherent cells, treatment with oxidants can induce cell death which will cause cell to detach from the coverslip, thus it is advisable to treat cells with sub-lethal doses. The issue does not exist in cells growing in suspension (e.g., Jurkat).

### Example 4

### Study of Thiol Oxidation for a Model Disease and for a Protein of Interest

### Material and Methods

### Rotenone Treatment.

Lewis rats were treated with rotenone as previously described (Betarbet, R.; Sherer, T. B.; MacKenzie, G.; Garcia-Osuna, M.; Panov, A. V.; Greenamyre, J. T. Chronic systemic pesticide exposure reproduces features of Parkinson's disease. Nat Neurosci 3:1301-1306; 2000).

Primary antibody labeling. The process was carried out using the AlexaFluor 488 antibody labeling kit (Invitrogen, Carlsbad, Calif.) according to the manufacturer's instructions. Briefly, the lyophilized primary antibody was resuspended in PBS. Sodium bicarbonate buffer (pH 8.5) was added 0.1M final. The antibody solution was incubated with the fluorophore for 1 hour. Excess, of unreacted dye was removed with a MICRO BIO-SPIN™ chromatography column 6 (Biorad, Hercules, Calif.).

Assessment of the alkyating efficiency of the thiol probes. SH-SY5Y human neuroblastoma cells were cultured according to the American Type Culture Collection (ATCC) directions. About 2·10⁶ cells were used for each thiol probe labeling. Cultured cells were harvested with trypsin and resuspended in PBS. To solubilize the plasma membrane, cells were incubated 10 minutes at room temperature in 5 μM digitonin, with gentle shaking. After incubation, permeabilization of the plasma membrane was confirmed counterstaining a fraction of the cells with trypan blue, which will penetrate only in permeabilized cells. In all the experiments, at least 98% of the cells were permeabilized. Free thiols were blocked by incubating the cells with 100 mM N-ethylmaleimide (NEM), 100 mM iodoacetamide (IAM) in Tris HCl 0.1 M, pH 6.8 for 15 minutes. The blocking and the labeling alkylating reactions were carried out at pH 6.8 because the reaction of maleimide with thiols is more selective at a pH that is slightly lower than the physiological one (Britto, P. J.; Knipling, L.; Wolff, J. The local electrostatic environment determines cysteine reactivity of tubulin. J Biol Chem 277:29018-29027; 2002; Haugland, R. P.; Haugland, R. P. Handbook of fluorescent probes and research products. Eugene, Oreg.: Molecular Probes; 2002; and Hermanson, G. T. Bioconjugate techniques. San Diego: Academic Press; 1996).

To remove the excess of alkylating agents, cells were washed by spinning them down and resuspending them in PBS for three times. Disulfides were reduced with 25 mM Tris(2-carboxyethyl)phosphine hydrochloride (TCEP) in PBS for 20 minutes at room temperature. Cells were spun down and resuspended in alkylating solutions containing various thiol probes at a concentration of 0.5 mM final. 10 mM stock solutions were prepared according to the manufacturer's directions. After 1 hour incubation, cells were spun down and resuspended in SDS-electrophoresis loading buffer (50 mM TrisHCl pH 6.8, 2% SDS, 0.1% blue bromophenol, 10% glycerol); the samples were sonicated and heated to denature proteins. Proteins were separated with SDS-electrophoresis according to standard procedures. The gel was imaged with a UV transilluminator (Bio-Doc-IT imaging system, UVP, Upland, Calif.). To confirm equal protein loading, the gels were counterstained with 0.2% G-250 Coomassie (SERVA, Heidelberg, Germany) dissolved in 35% methanol, 3.5% ortophosphoric acid, 1.3M ammonium sulfate.

Spectrophotometric analysis. BSA was dissolved in PBS at a final concentration of 10 mg/ml and CPM in Tris HCl (0.1 M, pH 6.8) was added to a final concentration of 0.5 mM. After 20 minutes of incubation at room temperature with gentle shaking, unreacted CPM was removed with gel-filtration. Emission fluorescence spectra were acquired with a RF-5301 PC spectrofluorophotometer. To study FRET between CPM and Alexa-488, an Alexa-488 conjugated IgG was modified with CPM as above. The spectra were acquired as above.

Histological labeling of oxidized thiols. Brains were fixed in 4% paraformaldehyde and processed as described (Betarbet, R.; Sherer, T. B.; MacKenzie, G.; Garcia-Osuna, M.; Panov, A. V.; Greenamyre, J. T. Chronic systemic pesticide exposure reproduces features of Parkinson's disease. Nat Neurosci 3:1301-1306; 2000). After cutting with a freezing microtome, sections were treated immediately in alkylating buffer (100 mM IAM, 100 mM NEM, TrisHCl, pH 6.8) to block free thiols. For cell culture studies, cells were seeded and cultured on coverslips, then fixed in 4% paraformaldehyde, 0.02% Triton-X100 supplemented with 100 mM IAM, 100 mM NEM to block free thiols. After three washes in PBS—10 minutes at room temperature—disulfides were reduced with 25 mM TCEP in PBS for 20 minutes. TCEP was omitted in the control reaction. The samples were labeled with CPM (0.5 mM final) in TrisHCl (0.1 M, pH 6.8) and then washed three times, for 10 minutes, in PBS.

Following the thiol labeling, immunohistochemistry was performed according to standard procedures. Briefly, after a blocking step in 10% serum, samples were incubated over-night with primary antibody (tyrosine hydroxylase, Mab318, Chemicon, Temecula, Calif., 1:2000; OX42, MCA 275G AbD Serotec, Oxford, UK, 1:150; GFAP, Mab 3402, Chemicon, Temecula, Calif., 1:1000). Fluorescently labeled secondary antibodies were used for detection. For FRET analysis, the thioredoxin antibody (R&D Systems, Minneapolis, Minn., 1:500) was directly labeled with AlexaFluor 488, as described above.

Images were acquired using a laser scanning confocal microscope. The microscope was equipped with a spectral detector technology that provides precise wavelength separation of the emitted light, preventing light leakage between different fluorophores. The following detection ranges were used for each fluorophore: 450 nm-480 nm for UV probes; 515 nm-535 nm for Alexa488; 560 nm-574 nm for Cy3. The acquisition parameters were set in the control reaction performed without TCEP and remained constant for all the consequent analyses.

Direct FRET was detected by reading the acceptor emission (FITC derivative conjugated antibody) at 520 nm while exciting the donor (CPM) with the 405 nm laser. Alternatively, FRET was detected by acquiring the donor (CPM) emission before and after photobleaching of the acceptor fluorophore (Alexa488) with the 488 nm laser. FRET efficiency (F_{eff}) was calculated by the confocal software (version 1.5) according to the equation F_{eff}=1−(emₚᵣₑ/emₚₒₛₜ), where F_{eff}=FRET efficiency, emₚᵣₑ=donor emission prebleaching, emₚₒₛₜ=donor emission post bleaching.

Analysis of disulfides in cells incubated in redox buffers. Cells were cultured in 96-well plates at a density of 5×10⁴ cells per well. Redox buffers were prepared as previously described, with minor modifications (W. H. Watson et al., Oxidation of nuclear thioredoxin during oxidative stress. FEBS Lett 543: 144-147 (2003)). Briefly, different amounts of oxidized and reduced glutathione (GSH, where 2GSH→GSSG) were combined in 4% paraformaldehyde (0.5 M TrisHCl pH 7.4) and 0.02% Triton X-100 to obtain solutions at the desired redox potential. Solutions were obtained at various redox potentials: −150 mV, with GSH 5 mM and GSSG 27.77 mM; −180 mV with GSH 10 mM, and GSSG 18.72 mM; −210 mV with GSH 20 mM and GSSG 4.14 mM; −240 mV with GSH 40 mM and GSSG 1.6 mM; −270 mV with GSH 40 mM and GSSG 0.15 mM; −300 mV with GSH 40 mM and GSSG 0.015 mM. Cells were fixed in the redox buffered paraformaldehyde for 1 hour.

Thiols were stained as above; nuclei were counterstained with Sytox green 0.5 μM (Invitrogen, Carlsbad, Calif.) according to the manufacturer's instructions. Cells were imaged with a microplate spectrofluorometer (Spectramax Gemini EM, Molecular Devices, Sunnyvale, Calif.). Thiol levels were expressed as the ratio of CPM staining to Sytox green signal.

Western blot analysis to detect proteins with oxidized cysteines. Cells were lysed in alkylating buffer (0.1M TrisHCl pH 6.8, 1% SDS, 100 mM IAM, 100 mM NEM). The lysate was sonicated on ice to disrupt high molecular weight DNA and incubated 30 minutes at room temperature to allow total alkylation of free Cys residues. 200 μg of alkylated proteins were precipitated with 5 volumes of ice-cold acetone and resuspended in 50 microliters of Tris-SDS buffer (0.1M TrisHCl, pH 7.4, 1% SDS). Disulfides were reduced by adding TCEP 5 mM final concentration. The samples were incubated 20 minutes at room temperature to allow complete reduction of disulfides to thiols. TCEP was removed with Micro Bio-spin Chromatography columns 6 (BIORAD, Hercules, Calif.). After the filtration step, the samples were in about 50 μL of 10 mM TrisHCl pH 7.4 (volume was adjusted to 50 μL as necessary). SDS was added to a 1% final concentration.

To label the previously oxidized (and now reduced) cysteines, polyethylene-glycol-conjugated maleimide (molecular weight 10 kDa, SunBio, Orinda, Calif.) was added to 50 micrograms of proteins, 1 mM final concentration, from a 10 mM stock in Tris-SDS buffer. After 1 hour incubation at room temperature, samples were precipitated with ice cold acetone and resuspended in 50 μL of SDS electrophoresis loading buffer. 10 μL (about 10 μg) were used for western blot analysis. The thioredoxin antibody (R&D Systems, Minneapolis, Minn.) was used at 1:1000 dilution.

Preparation of affinity resin. Activated thiol-sepharose resin 4B (Sigma, St. Louis, Mo., USA) was used in previous studies to purify thiol-containing proteins (Egorov, T. A.; Svenson, A.; Ryden, L.; Carlsson, J. A rapid and specific method for isolation of thiol-containing peptides from large proteins by thiol-disulfide exchange on a solid support. Proc Natl Acad Sci USA 72:3029-3033; 1975); the resin extent of binding was 1 micromol of thiols per mL of medium. The resin rehydrated in milliQ-grade distilled water, 30 minutes, gently shaking at 4° C. After centrifugation (5 minutes, 500×g), the sedimented resin was resuspended in 10 volumes of Tris-SDS buffer (0.1M TrisHCl, pH 7.4, SDS 1%) and incubated 5 minutes, gently shaking at room temperature. This step was repeated three times. The resin was resuspended at a final concentration of 150 mg/ml.

Purification of oxidized proteins. Brains were collected and different regions were micro-dissected, separated and homogenized in 0.32 M sucrose containing 30 mM TrisHCl (pH 7.4) and 1 mM EDTA, on ice, using a Teflon glass homogenizer. Samples were spun (900×g, 10 mM, 4° C.) to remove nuclei and debris and the supernatant was spun again (10000×g, 15 mM, 4° C.). The pellet, which represents an enriched mitochondrial fraction, was use for the subsequent analysis. Proteins (0.5 mg) were resuspended in Tris-SDS buffer at the final concentration of 1 mg/ml. Proteins were denatured by heating the solution at 70° C. for 5 minutes. NEM and IAM were added at the final concentration of 100 mM each. The mixture was incubated 30 minutes at 37° C. and precipitated with 5 volumes of ice-cold acetone for 1 hour at −80° C. After spinning (25 mM, 3200×g, 4° C.), the pellet was resuspended in 1 ml of Tris-SDS buffer As a pre-clearing step, the sample was then incubated with the thiol-sepharose medium (300 microliters, about 50 mg of dry resin) for 2 hours at room temperature. After centrifugation (5 minutes, 500×g, room temperature), the supernatant was saved and TCEP (25 mM final concentration) was added to reduce cysteines previously engaged in disulfides. The mixture was incubated 15 minutes at room temperature. A control reaction was performed by incubating proteins without TCEP.

Proteins were then precipitated as above and resuspended in 4 ml of Tris-SDS. Affinity purification was performed by incubating for 2 hours at room temperature, the mixture with 1 ml of equilibrated resin (about 150 mg of dry resin). To wash the resin after incubation, the sample was spun for 5 mM at 500×g, room temperature. The supernatant (unbound) was saved, and the sedimented resin was resuspended in 10 volumes of Tris-SDS, shaken 3 minutes at room temperature, and spun again. A total of 3 washes were performed. Bound proteins were eluted by incubating the resin (5 minutes, shaking, room temperature) with Tris-SDS buffer containing 100 mM DTT. After centrifugation (5 minutes, 500 g, RT), the supernatant (eluate) was saved and the sedimented resin was incubated once more with the eluting buffer. After centrifugation, the proteins in the pooled supernatants and in the unbound fraction were precipitated with acetone and used for electrophoresis.

### Results

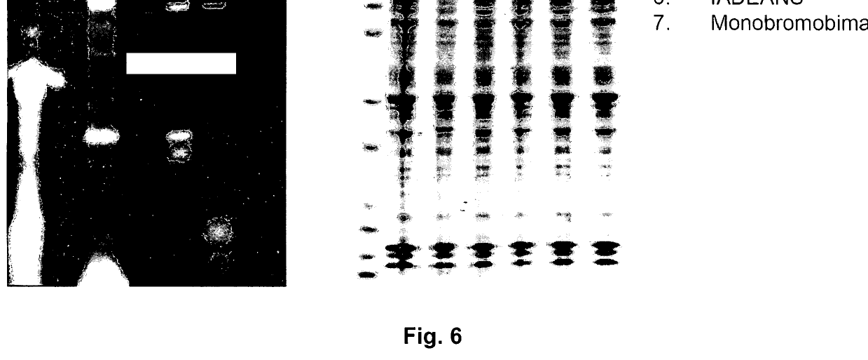

Oxidized disulfides can be modified through a multi-step process as shown in FIG. 5, involving the alkylation of free thiols, the reduction of disulfides and the modification of the newly formed free thiols with fluorescent probes. In this study, an important new negative control—in which the reducing step was omitted from the protocol—was included. Under these conditions, the signal detected by the instrument was due to background noise and was used to set the zero level of the acquisition parameters.

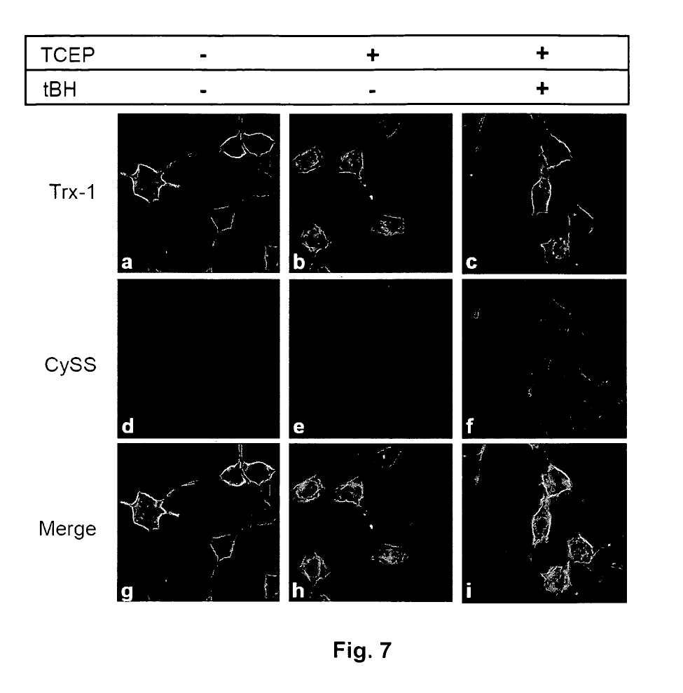

After very gentle solubilization of the plasma membrane, intact cells were labeled with five different probes that differ by thiol-reactive functional group or fluorescent conjugate. The labeling capacity varied greatly, depending upon the functional group and its conjugate. FIG. 6 shows that 7-Diethylamino-3-(4′-maleimidylphenyl)-4-methylcoumarin (CPM), a derivative of maleimide, was found to be the most effective molecule and, therefore, it is the one that was used for subsequent studies.

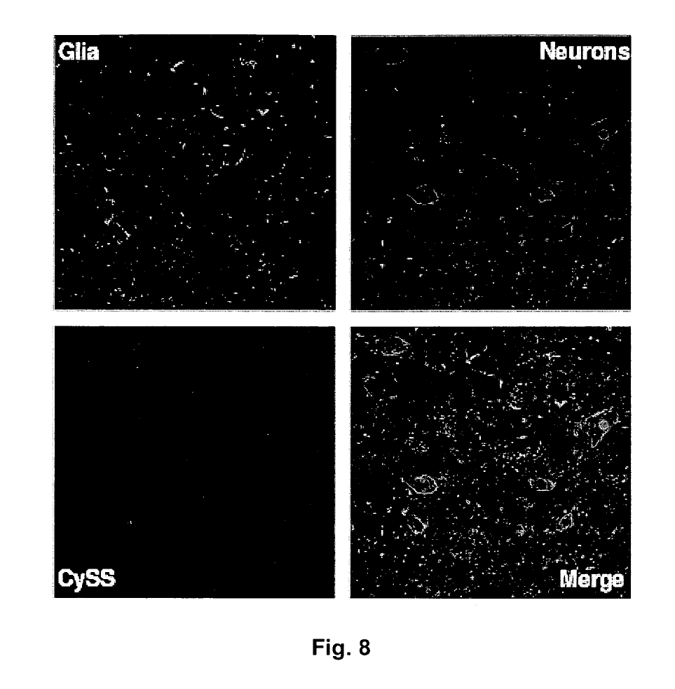

In order to assess the ability of CPM to detect variations in disulfides in situ as well as to generate a calibration curve, disulfide staining was applied to cells that were fixed in paraformaldehyde solutions at known redox potentials. CPM emission increased linearly over a wide range of redox potentials, from about −270 to −150 mV. Importantly, thiol labeling with CPM could detect variations in the levels of oxidized thiols in cells that were treated with tert-butyl-hydroperoxide (tBH), an oxidant commonly used in the study of thiol redox state (Watson, W. H.; Jones, D. P. Oxidation of nuclear thioredoxin during oxidative stress. FEBS Lett 543:144-147; 2003), and then fixed. In FIG. 7, panel (i) shows the colocalization of oxidized thiols (blue) and thioredoxin (green) in the presence of tBH.

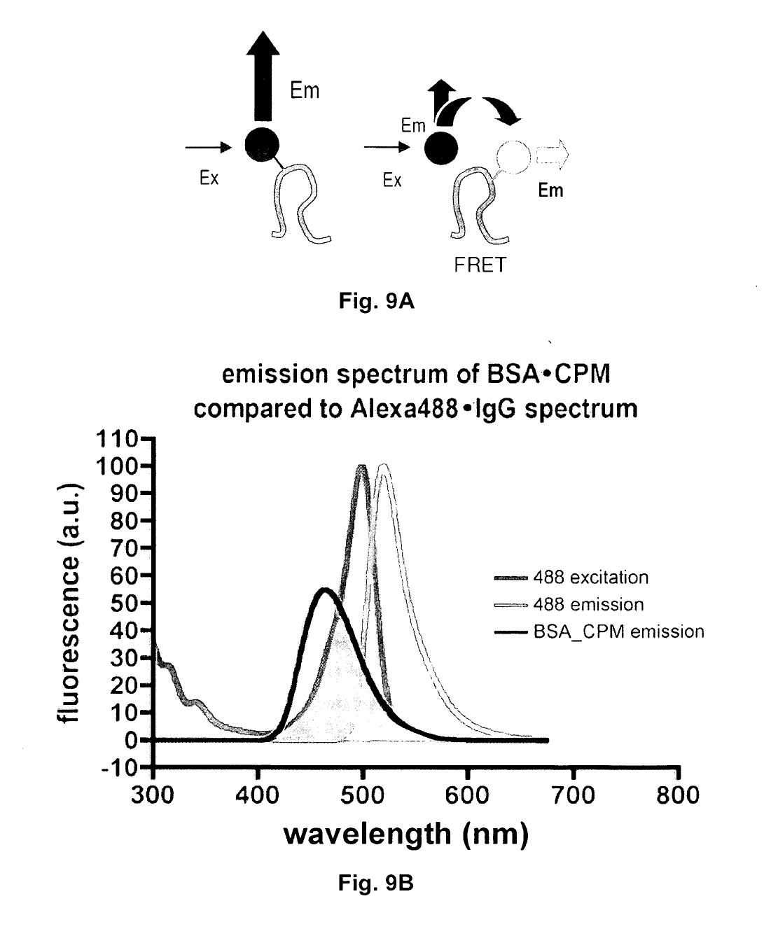

Chronic treatment of rats with rotenone to model Parkinson's disease induces oxidative stress and protein carbonylation. Initial studies—where proteins with oxidized thiols in the form of disulfides were purified by thiol-affinity chromatography—indicated a marked increase in thiol oxidation in mitochondria isolated from the substantia nigra of rotenone treated rats. Therefore, the methods described herein were used to define the particular area of the midbrain, as well as the specific cell type—e.g., neurons or glia—affected by rotenone-induced oxidation. FIG. 8 shows disulfide labeling used in combination with classical immunohistochemical staining for neuronal and glial markers. The disulfide labeling indicates that thiol oxidation occurs primarily in dopaminergic neurons of the substantia nigra.

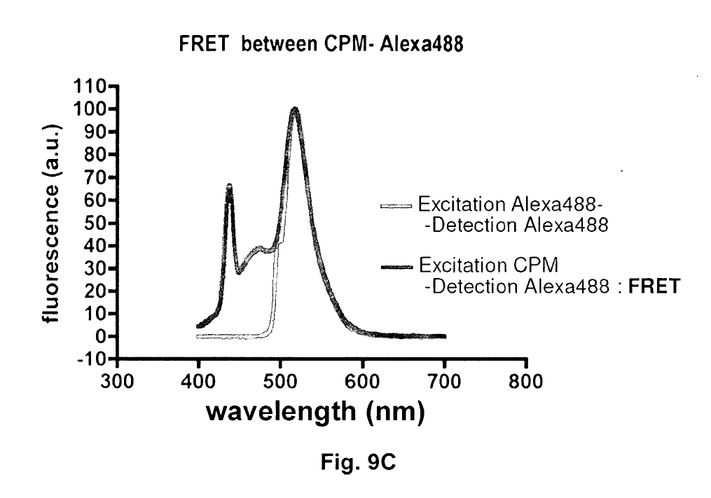

Such methods were also used to study thiol oxidation specifically in a protein of interest. This issue can be addressed investigating the fluorescence resonance energy transfer (FRET) between two different fluorophores. FRET occurs only if the two fluorophores are in close proximity (10-100 μ). As shown in FIG. 9A, FRET can provide information on whether the two probes are on the same (or very closely interacting) protein(s).

A necessary requisite for FRET to occur is that the emission spectrum of the donor molecule should overlap with the excitation spectrum of the acceptor. In this case, the donor molecule is CPM. It was found that FITC or Alexa488 dyes satisfied this prerequisite. FIG. 9B shows the overlap between the emission spectrum of CPM conjugated to BSA (dark blue) and the excitation spectrum of Alexa488 conjugated to IgG (gray area). To prove that FRET can occur between CPM and Alexa488, if present on the same protein, we conjugated CPM to an Alexa488-labeled IgG. FIG. 9C shows that the excitation of CPM resulted in indirect emission of fluorescence by Alexa488.

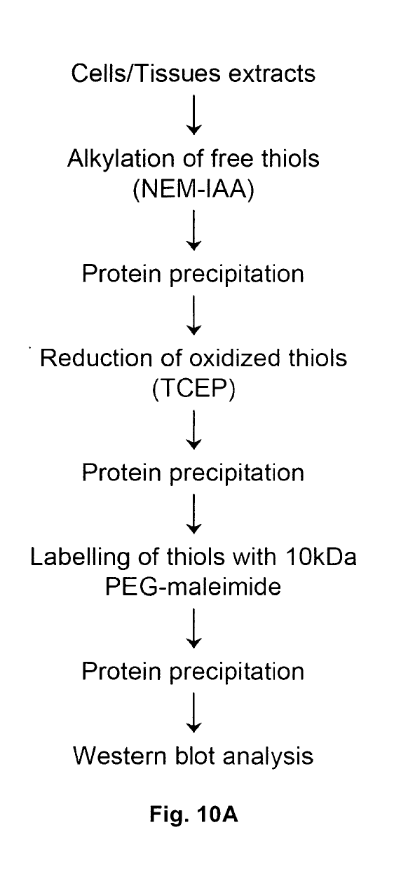

To determine if thiol staining used in combination with classical immunohistochemical staining and FRET could detect oxidation in a protein of interest, oxidation in thioredoxin-1 (trx-1) following treatment of cells with tBH was studied as a model system. Initially, it was established under what experimental conditions trx-1 was oxidized as shown in FIG. 10A.

In agreement with previous studies (Watson, W. H.; Jones, D. P. Oxidation of nuclear thioredoxin during oxidative stress. FEBS Lett 543:144-147; 2003), it was found that low millimolar concentrations of tBH quickly induced the formation of disulfides in trx-1 as shown in FIG. 10B. In this case, the thiols were labeled with maleimide conjugated to 10 kDa PEG and proteins were separated by SDS-PAGE. Thus, for each thiol group labeled with the conjugate, the mobility of the protein shifted by 10 kDa. Therefore, using a homogenate, one can create a “redox-ladder” of a protein of interest under different experimental conditions.

These oxidation conditions were used to study FRET between thiol-bound CPM and a specific Alexa488-conjugated anti-Trx-1 primary antibody. The bottom panel of FIG. 10C shows a clear increase in the signal for tBH treated cells, indicating thiol oxidation of Trx-1. As expected, the control reaction shown in the top panel of FIG. 10C, in which the reducing step was omitted, did not provide any signal.

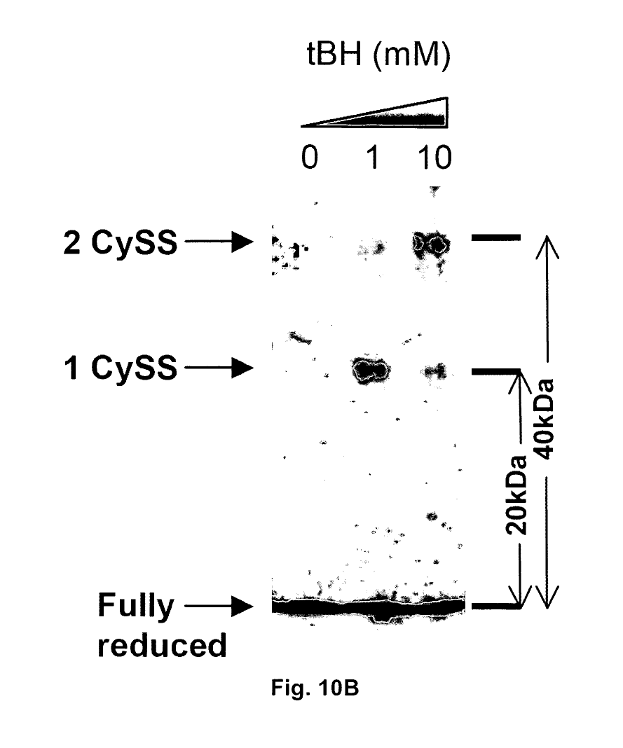

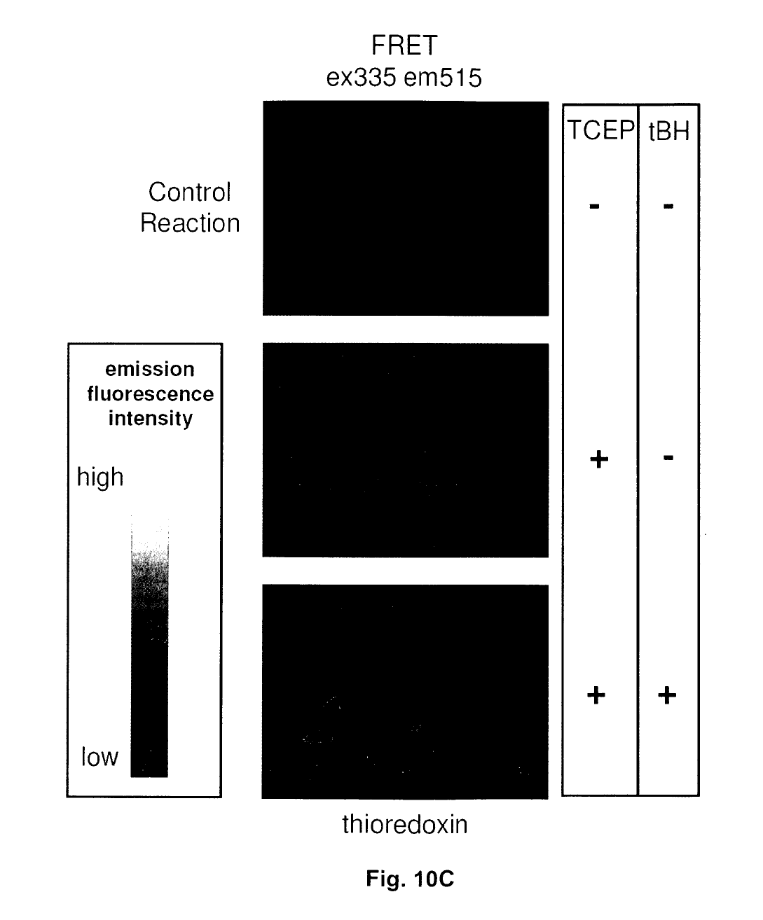

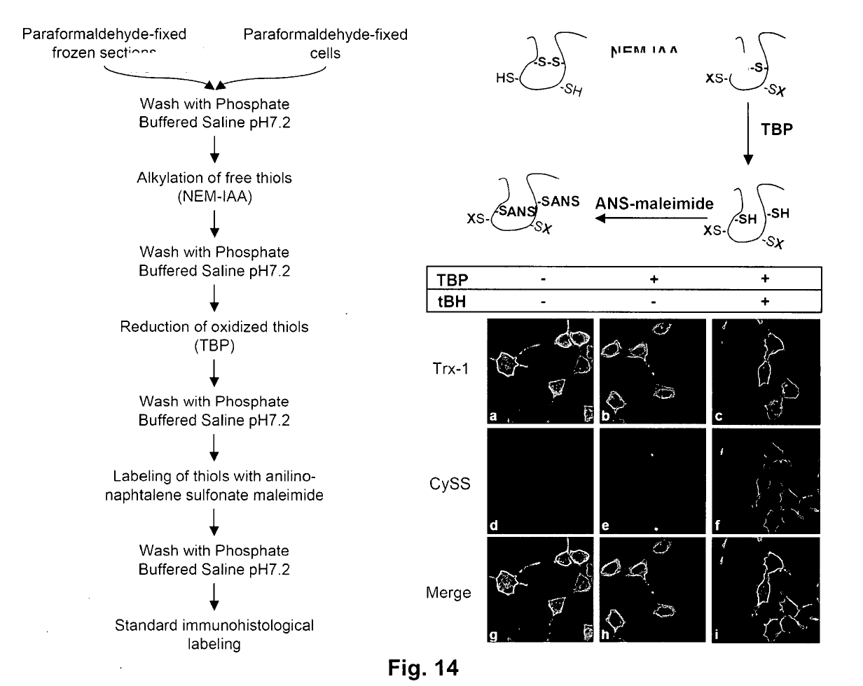

These findings were validated when FRET was analyzed through the acceptor photobleaching approach (Karpova, T. S.; Baumann, C. T.; He, L.; Wu, X.; Grammer, A.; Lipsky, P.; Hager, G. L.; McNally, J. G. Fluorescence resonance energy transfer from cyan to yellow fluorescent protein detected by acceptor photobleaching using confocal microscopy and a single laser. J Microsc 209:56-70; 2003), as shown in FIG. 11. Identical results were obtained when MIANS and FITC—two fluorophores similar to CPM and Alexa488—were used to label respectively thiols and the antibody (data not shown). As a final control, beta-synuclein—a protein without cysteines in its primary sequence—was studied. As shown in FIG. 16, the protein was negative for thiol groupsd. As shown in FIG. 12, FRET was not detected after tBH treatment despite a stimulated emission of the Alexa488 conjugated primary antibody.

When applied to the rotenone model of Parkinson's disease, which is based on the chronic administration of an inhibitor of mitochondrial respiratory complex I, it was shown that thiol oxidation occurs specifically in dopaminergic (DA) neurons of the substantia nigra. Importantly, these are the neurons that are predominantly lost in Parkinson's disease (PD) itself; therefore, these data establish a direct correlation between thiol oxidation and DA neuron loss. The detection of high levels of oxidized thiols in DA neurons is consistent with previous reports and is in agreement with the notion that DA neurons are particularly prone to oxidation due to increased hydrogen peroxide production resulting from monoamine oxidase activity. Besides its application to studies of the brain, this technique can be applied in all those physio-pathological situations—such as cancer, intoxication and infectious disease—in which oxidative imbalance plays a role.

To detect thiol oxidation in a protein of interest, the proximity of the thiol probe to a labeled antibody specific for Trx-1 was studied. Because FRET occurs, the distance between the fluorophores needs to be between 20-80 Å; this distance is comparable to the average size of proteins. However, other key factors as the spectral properties and the orientation of the fluorophores contribute to the occurrence of FRET. Although the fluorophores can be carefully selected to optimize the spectral properties for FRET, the orientation of the fluorophores—once on their target—cannot be controlled easily. It is possible that the location of the epitope recognized by the antibody, its distance from the labeled thiols and the orientation of the antibody after epitope recognition would influence the performance of FRET. Therefore, optimization of the experimental settings using different clones or types of antibodies might be required. In certain examples it was found that polyclonal antibodies worked better than monoclonal antibodies, perhaps because they constitute a pool of antibodies each recognizing a different epitope and therefore increasing the chances of labeling an optimal epitope for FRET. Variability between different monoclonal antibodies was observed (data not shown).

### Example 5

### Detection of Oxidation in Tissues

Histological brain sections are obtained from animals treated with rotenone or PQ. Rotenone and PQ can be administered as described (Betarbet, R, Sherer, T B, MacKenzie, G, Garcia-Osuna, M, Panov, A V and Greenamyre, J T, (2000) Chronic systemic pesticide exposure reproduces features of Parkinson's disease. Nat Neurosci 3: 1301-6 and McCormack, A L, Atienza, J G, Johnston, L C, Andersen, J K, Vu, S and Di Monte, D A, (2005) Role of oxidative stress in paraquat-induced dopaminergic cell degeneration. J Neurochem 93: 1030-7). Sections from fixed brains are analyzed for disulfides (CPM), the proteins of interest (fluorophore-conjugated primary antibodies) and markers for specific cell types (neurons: NeuN; dopaminergis neurons: TH; microglia: OX-42; astrocytes: GFAP).

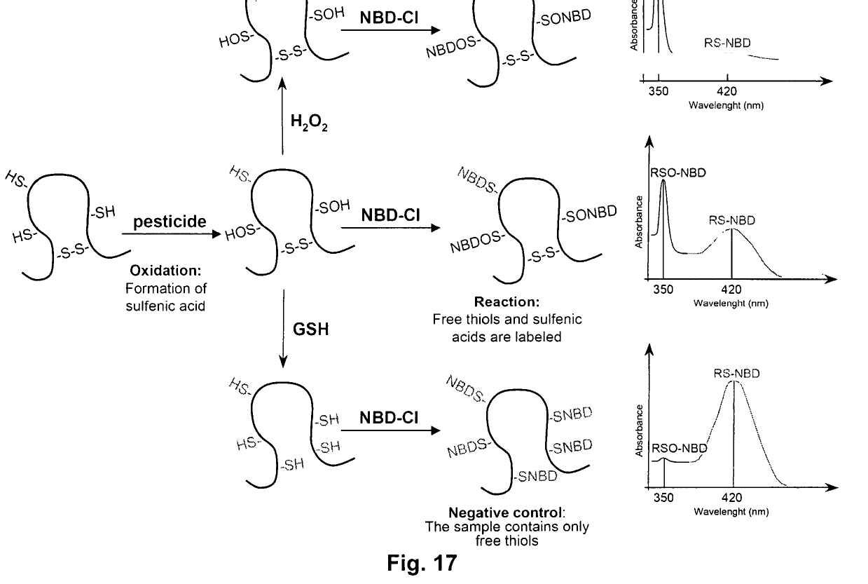

To detect carbonyls in fixed slices and perform microscopy studies, fluorescein-conjugated hydrazine is used (FITC-hydrazine, please see FIG. 19). As a positive control, carbonyl formation is induced by metal-catalyzed oxidation, treating the samples with iron (Fe³⁺) and ascorbate (Amici, A, Levine, R L, Tsai, L and Stadtman, E R, (1989) Conversion of amino acid residues in proteins and amino acid homopolymers to carbonyl derivatives by metal-catalyzed oxidation reactions. J Biol Chem 264: 3341-6 and Halliwell, B and Gutteridge, J M, (1990) Role of free radicals and catalytic metal ions in human disease: an overview. Methods Enzymol 186: 1-85). In the confocal studies, a negative control can be performed preincubating the fixed slices with an excess of DNPH.

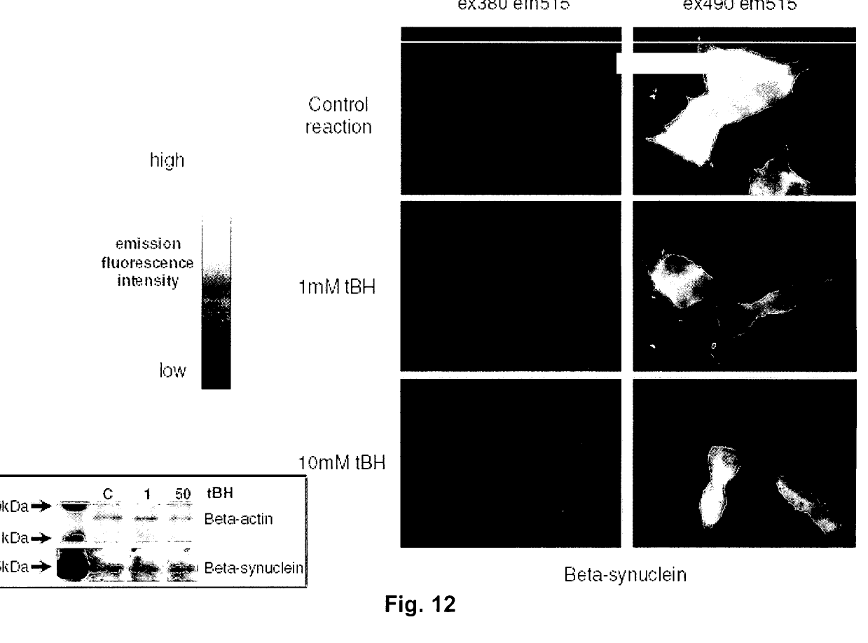

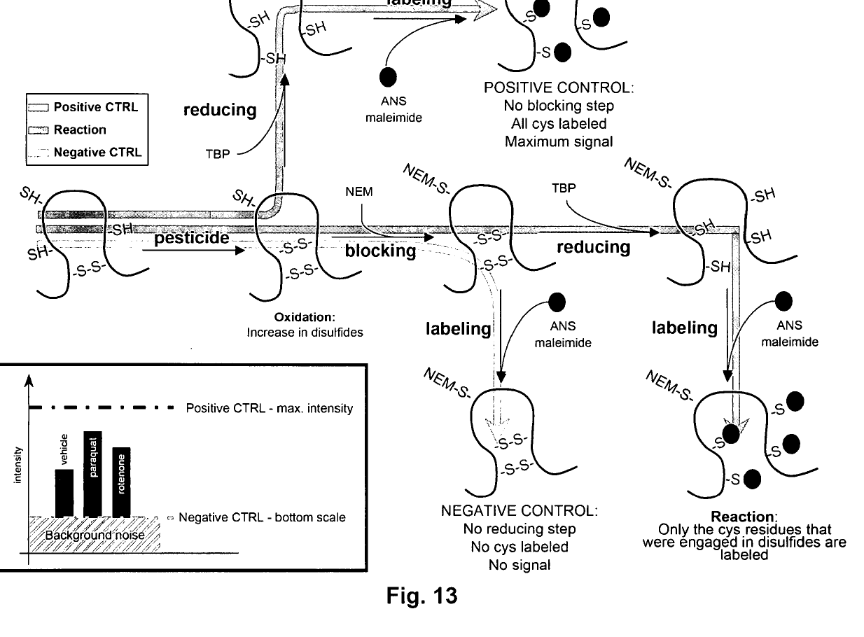

To determine oxidation of Cys to disulfides in a protein of interest, the FRET is detected between the cysteine tag (MIANS) and the labeled specific primary antibody (see FIGS. 14, 15 and 16), in association with staining specific for certain cell type (e.g., DA neurons or glia).

To detect cysteines oxidized to sulfenic acid the procedure(s) described herein is used. Sulfenic acid (RSOH) is the product of two electron oxidation of a thiol (RSH). Unlike disulfides, sulfenic acid is very unstable and extremely reactive; typically sulfenic acid can react with another thiol to form disulfides, or condense with a second sulfenic acid to form thiosulfinates (Claiborne, A, Miller, H, Parsonage, D and Ross, R P, (1993) Protein-sulfenic acid stabilization and function in enzyme catalysis and gene regulation. Faseb J 7: 1483-90). Therefore, even though sulfenic acid might be an earlier event, its stable form will likely be a disulfide. Sulfenic acid can be labeled using of 7-chloro-4-nitrobenzo-2-oxa-1,3-diazol (NBD-Cl) (Claiborne, A, Miller, H, Parsonage, D and Ross, R P, (1993) Protein-sulfenic acid stabilization and function in enzyme catalysis and gene regulation. Faseb J 7: 1483-90; Bryk, R, Griffin, P and Nathan, C, (2000) Peroxynitrite reductase activity of bacterial peroxiredoxins. Nature 407: 211-5; Carballal, S, Radi, R, Kirk, M C, Barnes, S, Freeman, B A and Alvarez, B, (2003) Sulfenic acid formation in human serum albumin by hydrogen peroxide and peroxynitrite. Biochemistry 42:9906-14; and Ellis, H R and Poole, L B, (1997) Novel application of 7-chloro-4-nitrobenzo-2-oxa-1,3-diazole to identify cysteine sulfenic acid in the AhpC component of alkyl hydroperoxide reductase. Biochemistry 36: 15013-8).

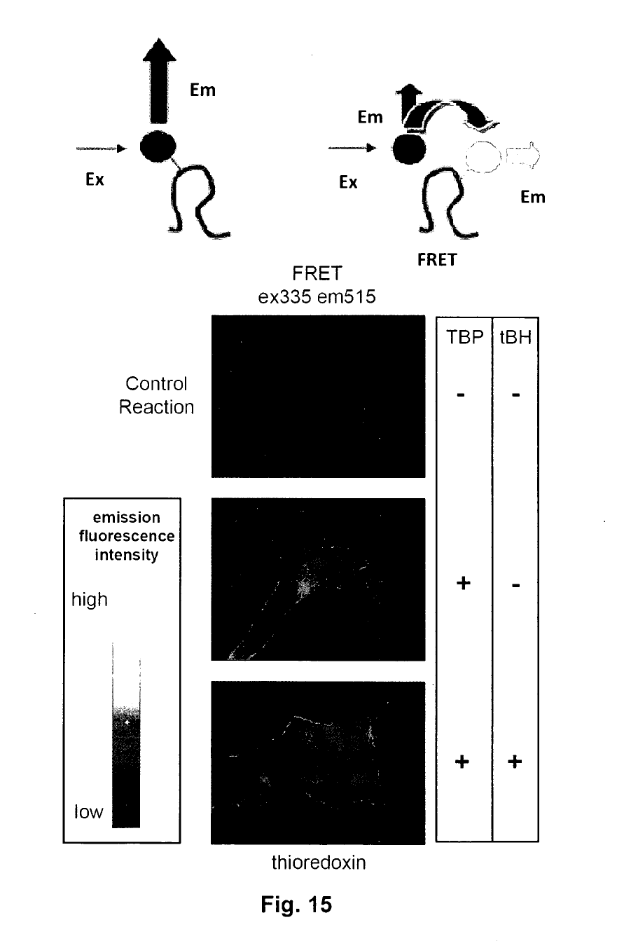

The sulfoxide adduct that is formed between sulfenic acid and NBD-Cl (RSO-NBD) absorbs at 350 nm NBD-Cl can react also with free thiols of Cys to form a thioether adduct (RS-NBD) that absorbs at 420 nm (FIG. 17). Therefore, the absorption spectra of the NBD-Cl adducts will provide information about the level of thiols—confirming the findings in (i)—as well as on the levels of sulfenic acids (Carballal, S, Radi, R, Kirk, M C, Barnes, S, Freeman, B A and Alvarez, B, (2003) Sulfenic acid formation in human serum albumin by hydrogen peroxide and peroxynitrite. Biochemistry 42:9906-14). As a positive control, formation of sulfenic acid is induced by treating the samples with hydrogen peroxide; negative controls are carried out by reducing the sulfenic acid with glutathione (Carballal, S, Radi, R, Kirk, M C, Barnes, S, Freeman, B A and Alvarez, B, (2003) Sulfenic acid formation in human serum albumin by hydrogen peroxide and peroxynitrite. Biochemistry 42:9906-14). As sulfenic acids are extremely reactive, slices are homogenized directly in the presence of NBD-Cl. Homogenization can be performed in denaturing conditions. Undesired adducts can be formed by NBD-Cl reaction with tyrosyl and amino groups at alkaline pH; thus, the reaction can be performed at neutral pH. Labeled samples are analyzed with a fluorescence spectrophotometer.

Also, tyrosine oxidation to nitrotyrosine is detected. Tyrosine can be modified to nitrotyrosine (NO-Tyr) by peroxynitrite (ONOO—), a short-lived reactive nitrogen species (RNS), which is generated by the reaction of nitric oxide and superoxide, a by-product of mitochondrial oxidative phosphorylation. Formation of NO-Tyr in proteins has been associated to glutathione depletion, mitochondrial dysfunction and PD (Bharath, S and Andersen, J K, (2005) Glutathione depletion in a midbrain-derived immortalized dopaminergic cell line results in limited tyrosine nitration of mitochondrial complex I subunits: implications for Parkinson's disease. Antioxid Redox Signal 7: 900-10; Giasson, B I, Duda, J E, Murray, I V, Chen, Q, Souza, J M, Hurtig, H I et al., (2000) Oxidative damage linked to neurodegeneration by selective alpha-synuclein nitration in synucleinopathy lesions. Science 290: 985-9; and Murray, J, Taylor, S W, Zhang, B, Ghosh, S S and Capaldi, R A, (2003) Oxidative damage to mitochondrial complex I due to peroxynitrite: identification of reactive tyrosines by mass spectrometry. J Biol Chem 278: 37223-30). NO-Tyr can be detected by immunological means, as different manufacturers have produced several specific antibodies Immunohistolgical and immunoblotting studies are used to detect NO-Tyr in organotypic slices after one, two, three, or four weeks of treatment with PQ or rotenone. A negative control is made by pre-incubating the antibodies with nitro-tyrosine; as an alternative strategy, nitrotyrosines can be reduced with repeated washes in dithionite (Viera, L, Ye, Y Z, Estevez, A G and Beckman, J S, (1999) Immunohistochemical methods to detect nitrotyrosine. Methods Enzymol 301: 373-81). As a positive control, the samples are treated with nitrating agents (e.g., ONOO—).

### Example 6

### Techniques for Determining Oxidative Modification

Thiol oxidation. Reversible oxidation of the thiol (SH) groups in cysteine residues of proteins plays crucial roles in redox biochemistry and enzymatic activity. Disulfide (S—S) bond formation is critical for normal secondary and tertiary protein structure. However, under conditions of oxidative stress, new intra- and inter-molecular disulfide bonds may form and cause enzyme inactivation or protein cross-linking. Although such thiol oxidation may be reversible, if the cellular redox status is compromised, it may lead to irreversible cellular dysfunction. As an early—and potentially reversible—oxidative event, thiol oxidation is of particular interest in pesticide intoxication and related pathologies, in particular PD, where there is a marked loss of reduced glutathione at the earliest stages (Dexter, D T, Sian, J, Rose, S, Hindmarsh, J G, Mann, V M, Cooper, J M et al., (1994) Indices of oxidative stress and mitochondrial function in individuals with incidental Lewy body disease. Ann Neurol 35: 38-44). For this reason, methods were developed to provide (i) unbiased proteomic approaches for identifying proteins by their acquisition of new disulfide bonds; (ii) a redox western blot technique to assess the oxidation state of specific proteins; (iii) a histochemical technique to identify regional, cellular and subcellular levels of disulfide bonds; and (iv) a method to apply fluorescence resonance energy transfer (FRET) so as to assess the oxidation state of specific proteins of interest in tissue sections or cells in culture.

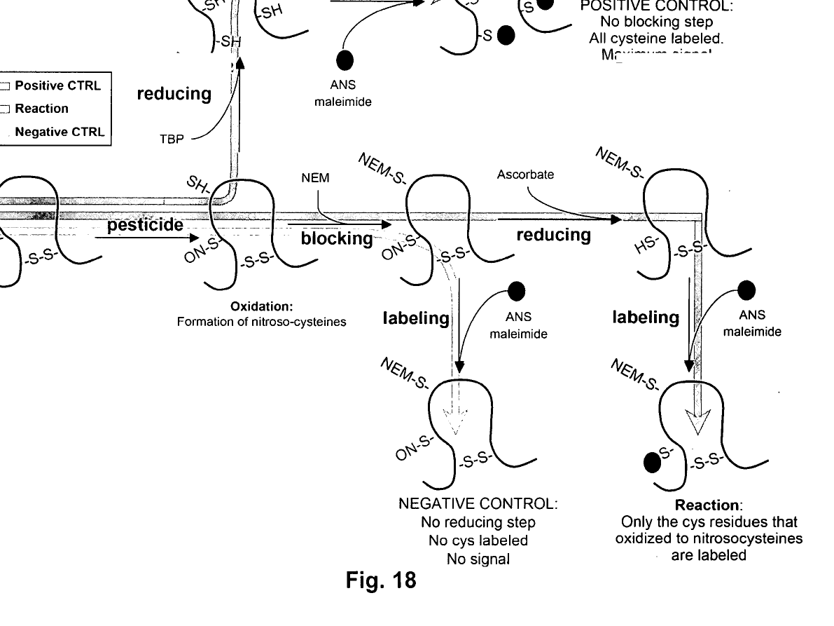

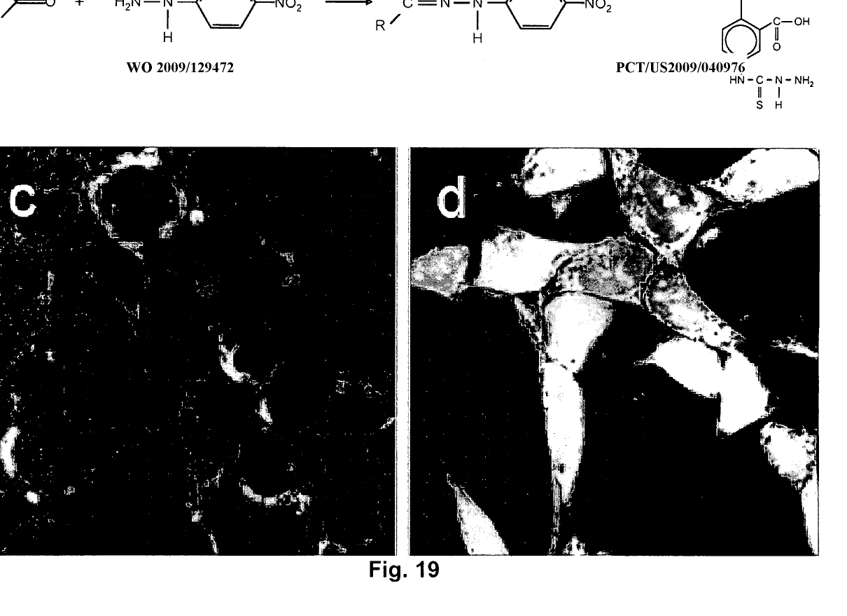

In the first technique (FIG. 20), a starting sample (e.g., purified mitochondria from the ventral midbrain of control and rotenone-treated rats) is homogenized and then free thiols are alkylated. The sample is then reduced, exposing what were previously disulfide bonds as free thiol groups. At this point, the sample can be run over a thiol affinity column and the eluate can be analyzed by SDS-PAGE and Coomassie staining. In an alternative strategy, after the reduction the sample can be reacted with ICAT (isotope coded affinity tag) reagents for mass spectrometry (Gygi, S P, Rist, B, Gerber, S A, Turecek, F, Gelb, M H and Aebersold, R, (1999) Quantitative analysis of complex protein mixtures using isotope-coded affinity tags. Nat Biotechnol 17: 994-9).

This technique allowed determination of a dramatic increase in oxidized proteins after exposure to rotenone and identified transferrin in mitochondria. Moreover, the ICAT reagents allowed identification of the oxidized thiol residue in transferrin as cys260.

An important control is to omit the reducing step after alkylation of the free thiols. In this so-called ‘control reaction’ there should be no free thiols available for affinity chromatography, ICAT labeling, or other reactions described below. For example, please see lane 6 of FIG. 20b.

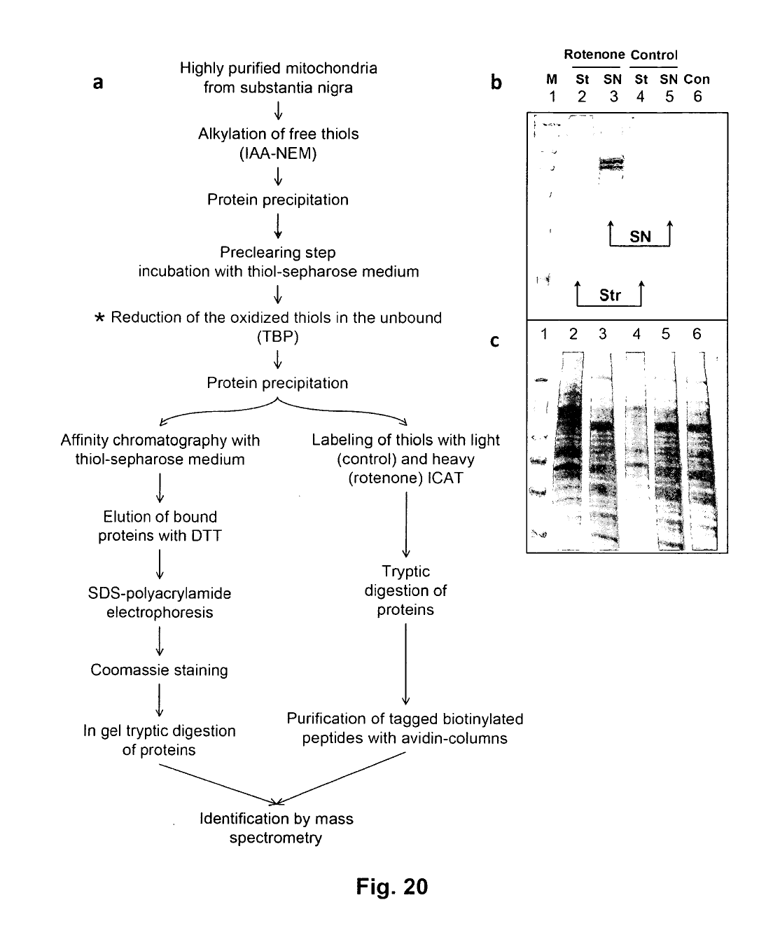

In the second technique (FIG. 21), the samples are processed to the point at the disulfides are reduced with tributylphosphine (TBP) and then reacted with the thiol-reactive reagent maleimide, which has been conjugated to 10 kDa PEG. Thus, for every free thiol available to react with maleimide-PEG, a protein's apparent MW will shift by 10 kDa. If the disulfide is intramolecular, there will be 2 thiols exposed and the MW will shift by 20 kDa.

The third technique uses a similar approach to label tissue sections or cells in culture, as outlined in FIG. 14. In this case, after the reduction step, the thiols are labeled with anilino-naphthaline-sulfonate maleimide (ANS), which emits in the fluorescence in the UV range.

For all of these related assays, omission of the reducing step with TBP provides a useful control. An additional ‘real life’ control is to attempt to thiol-label or do FRET on a protein with no cysteine thiol groups. For this purpose, alpha- and beta-synuclein are used, neither of which has a cysteine residue (FIGS. 12 and 16).

Protein carbonyls. Another oxidative modification of proteins is carbonyl formation. Typically, carbonyls are assayed by reacting protein homogenates with hydrazine compounds (e.g., dinitrophenylhydrazine; DNPH) and then measuring the reaction product spectrophotometrically, or with antibody-based techniques. We have used a fluorescent hydrazine compound to directly label carbonyls (FIG. 19). As with thiol labeling, this technique is amenable to FRET so that candidate proteins can be examined for carbonyl content and changes therein in pesticide intoxication.

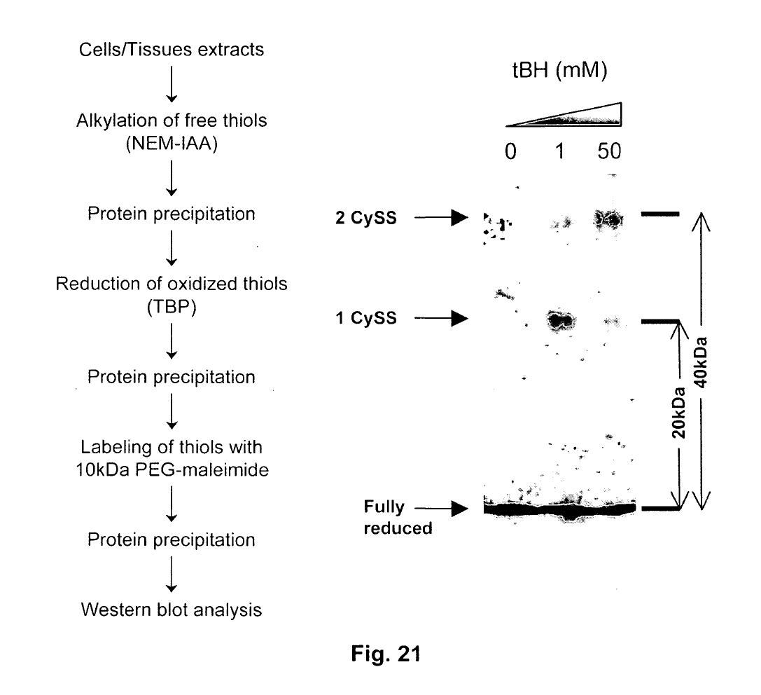

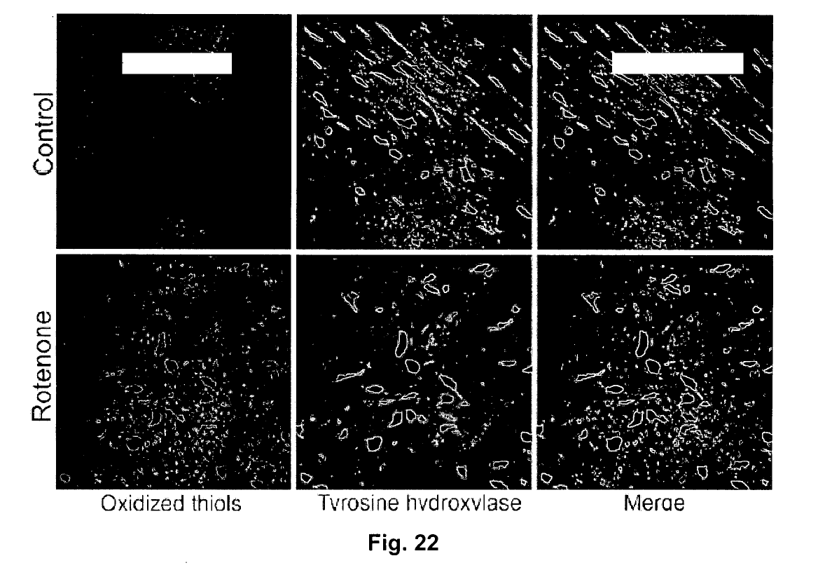

Oxidation of substantia nigra after rotenone treatment. As we have reported previously, rotenone causes oxidative damage in vivo (Sherer, T B, Betarbet, R, Testa, C M, Seo, B B, Richardson, J R, Kim, J H et al., (2003) Mechanism of toxicity in rotenone models of Parkinson's disease. J Neurosci 23: 10756-64). Our previous work has focused on protein carbonyls and could not provide anatomical resolution beyond the limits of our regional microdissections. Nevertheless, it was apparent that dopaminergic regions (ventral midbrain, striatum, olfactory bulb) incurred the most severe oxidative damage. With development of our new assays, it has been possible to examine with far greater anatomical resolution, the oxidative state of the brain after rotenone treatment. We find a remarkable and very selective increase in oxidized thiols in nigrostriatal dopaminergic neurons after rotenone (FIG. 22). In contrast to oxidized thiols (FIG. 22), the increase in carbonyls seen after rotenone is largely associated with astrocytic processes in the neuropil surrounding nigrostriatal dopamine neurons (FIG. 23). Thus the experiments demonstrated the ability to (1) labeled oxidized thiols in proteins in general; (2) identify oxidized thiols in specific cysteine residues of proteins of interest by ICAT labeling and mass spectrometry; (3) determine the oxidation status of oxidized thiols in candidate proteins of interest using redox western blots; (4) labeled oxidized thiols in histological specimens to identify cell types involved; (5) apply FRET to labeled histological specimens to identify the oxidation state of candidate proteins in cells or organelles of interest; and (6) label carbonylated proteins in cells/organelles of interest in histological specimens.

### Example 7

### Oxidative Events in Relation to Toxic Events Associated with PD

Various oxidative events are associated with Parkinson's disease (PD) or animal models of PD, such as dopaminergic cell loss, activation of astroglia and microglia, deposition of highly reactive iron, and accumulation of transferrin (trf). In order to study these oxidative events and to identify the key proteins involved, the oxidation status of proteins was probed before and after treating dopaminergic neurons with rotenone.

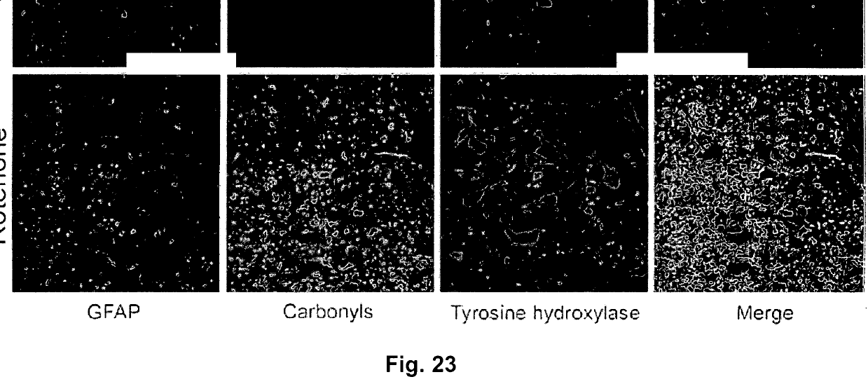

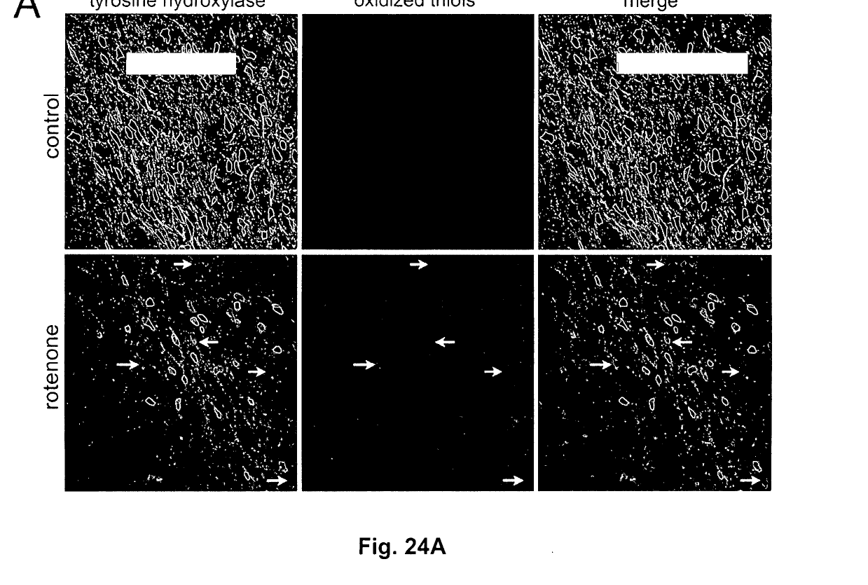

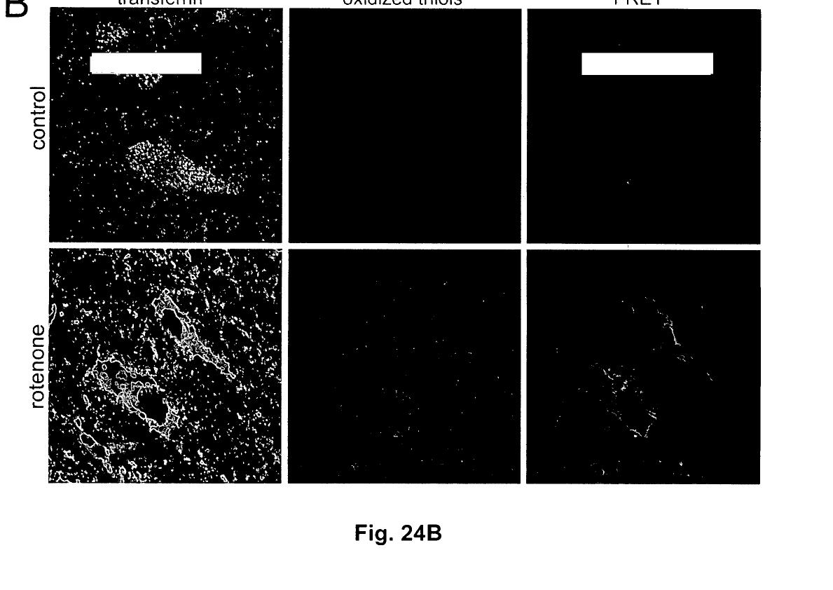

FIG. 24A shows dopaminergic cell loss upon treatment with rotenone. White arrows indicate neurons that lack tyrosine hydroxylase (TH) and possess oxidized thiols. Loss in TH, which is a key enzyme in dopamine metabolism, certainly reflects some neuronal damage and can be a measure of the toxic effect of the pesticide

FIG. 24B shows the accumulation of transferrin (trf) for cells treated with rotenone. By using FRET, the transferrin is shown to be colocalized with oxidized thiols. In contrast, dopaminergic neurons in the control show very little accumulation of trf.

FIG. 24C shows reducing and non-reducing Western blots of substantia nigra from a control animal (lanes labeled “C”) and a rotenone-treated animal (lanes labeled “R”) to detect thiol oxidation in transferrin. Upper panel, left, a reducing gel shows the transferrin band at the expected molecular weight, about 70 kDa. Upper panel, right, a non-reducing gel—where disulfides are preserved in the oxidized form—shows the monomeric form of transferrin migrating at the expected molecular weight (arrow) and a higher molecular weight form (arrow-head). This form—which is due to oxidized thiols, as it disappears in the reducing gel—is associated with chronic treatment with the pro-oxidant rotenone. In the lower panel, left and right, a loading control (39 kDa subunit of mitochondrial complex I) ensures that the initial amount of protein was the equal for all the lanes.

### Example 8

### Methods of Identifying which Proteins are Involved in the Thiol Oxidation Process

Provided herein is a method for testing the efficacy of a chemotherapeutic agent. First, the oxidation state of a tumor cell treated with the agent. Second, this obtained oxidation state is compared with the oxidation state of a tumor cell that is not treated with the agent. If the agent can induce the oxidation of thiols, then the agent would promote oxidative stress within the tumor and thereby prevent tumor growth.

This method can be used to determine the cellular effects of the chemotherapeutic agent, such as by identifying which proteins are involved in this thiol oxidation process. For example, FRET can be used to determine the presence of oxidized thiols in close proximity (from 10 Å to about 100 Å) with the protein of interest. This method can be used with tissue specimens that maintain the cellular integrity of an organ or tissue, where the cellular structure and extracellular matrix could be important for understanding tumor pathogenesis.

In addition, the method can be used to find a valid strategy for chemotherapy. This cell-based or sample-based method can be used to test the efficacy of drugs without the expense and complications of live animal studies. For example, this method can test combinatorial mixtures of chemotherapeutic agents, pharmaceutically acceptable carriers, buffers, and other drugs. In another example, this method can test the dosages of the drugs or the time-course treatment of the drugs.

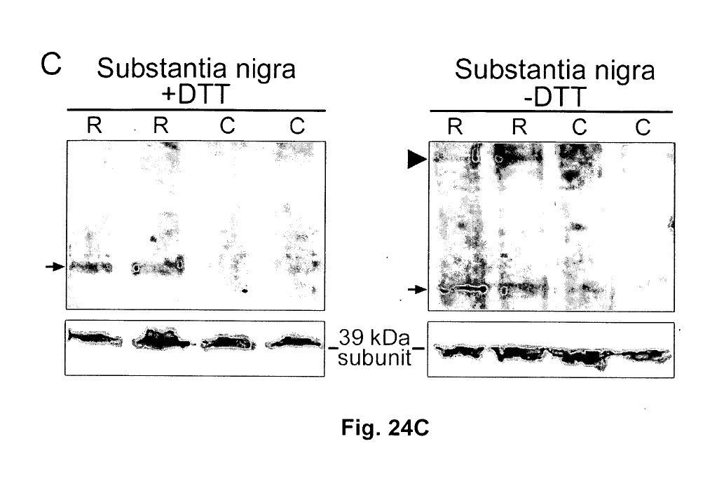

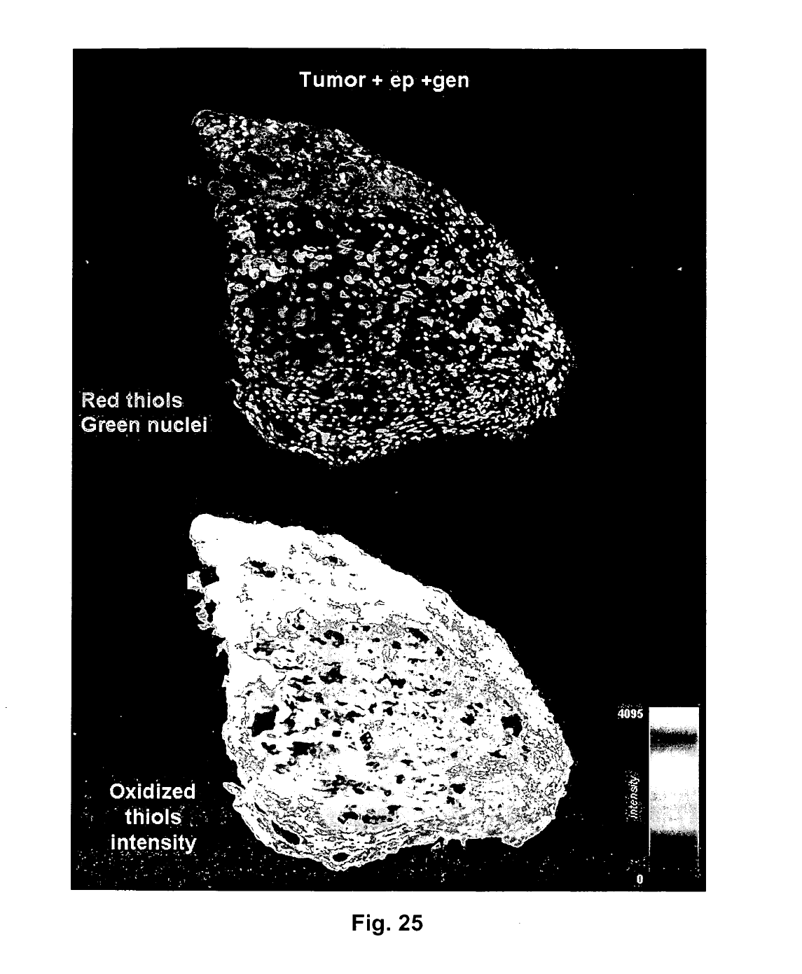

In one experiment, tumor cells were injected under the skin of nude mice. FIG. 25 shows a tumor that was treated with ethyl pyruvate and gentamicin. FIG. 26 shows tumor tissues treated with various agents, such as one of Ringer's solution, gentamicin, oxaliplatin, ethyl pyruvate, or melphalan.

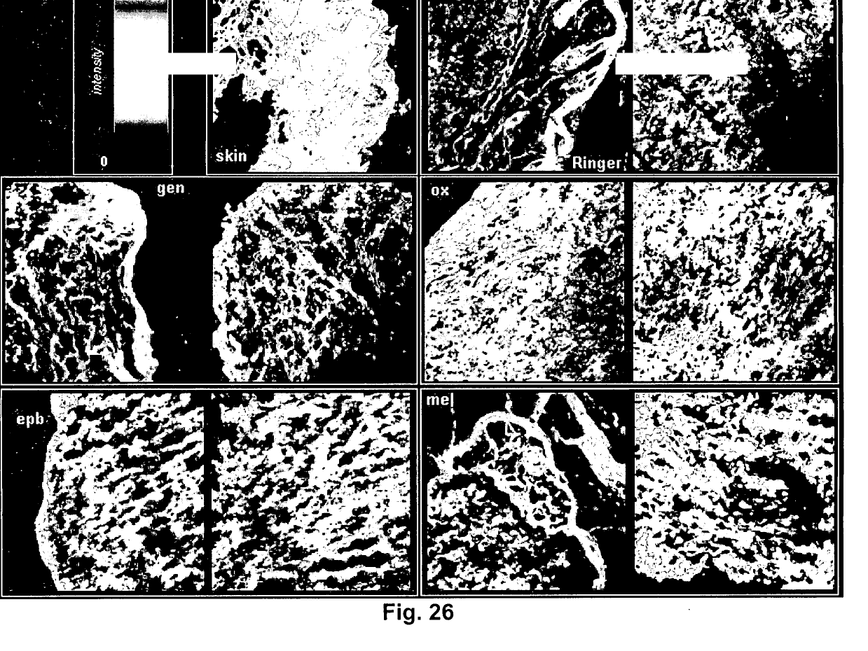

FIG. 27 shows tumor tissues that were treated with a combination of agents, where the highest level of oxidation was induced by the combination of ethyl pyruvate and oxaliplatin.

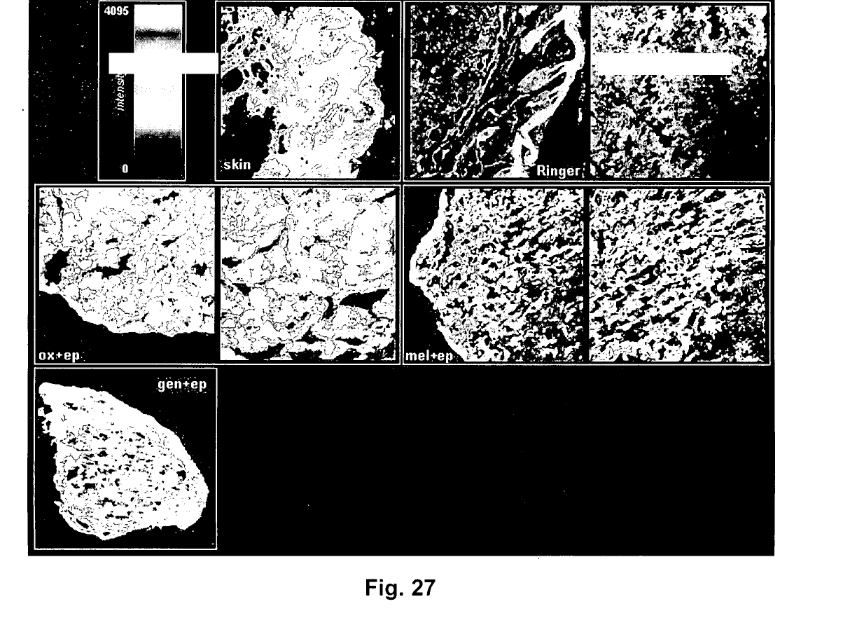

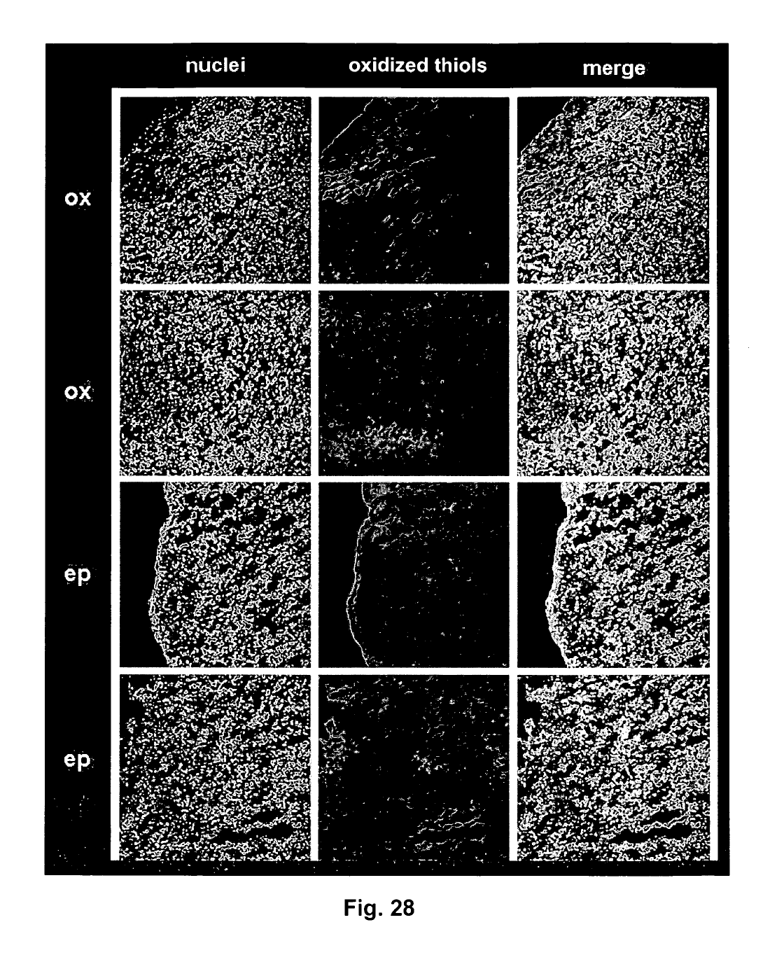

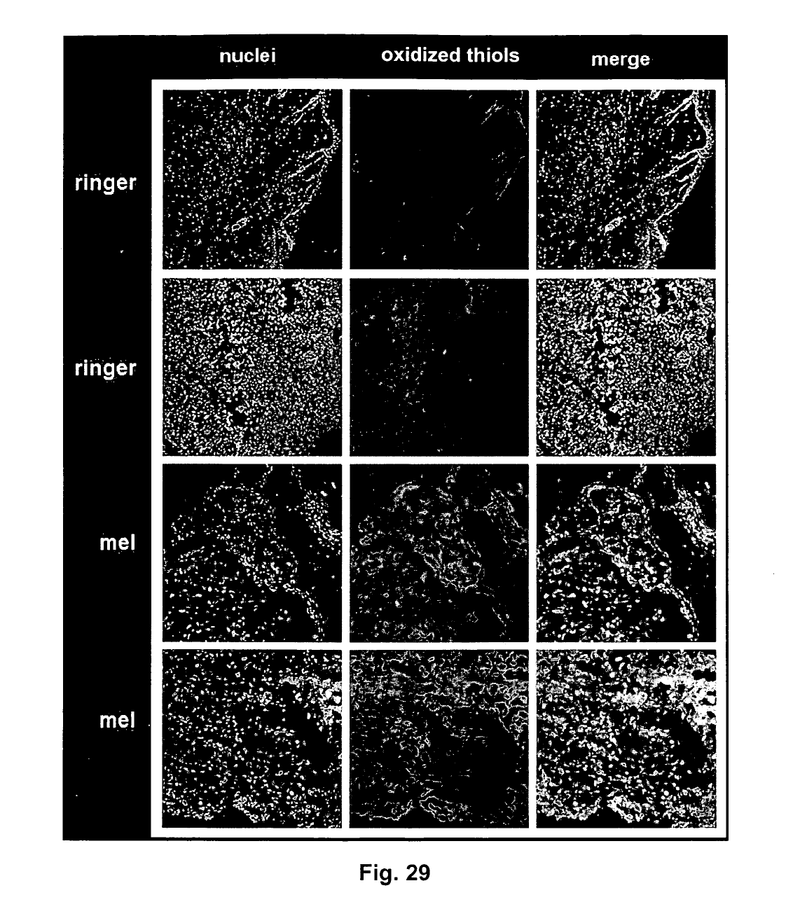

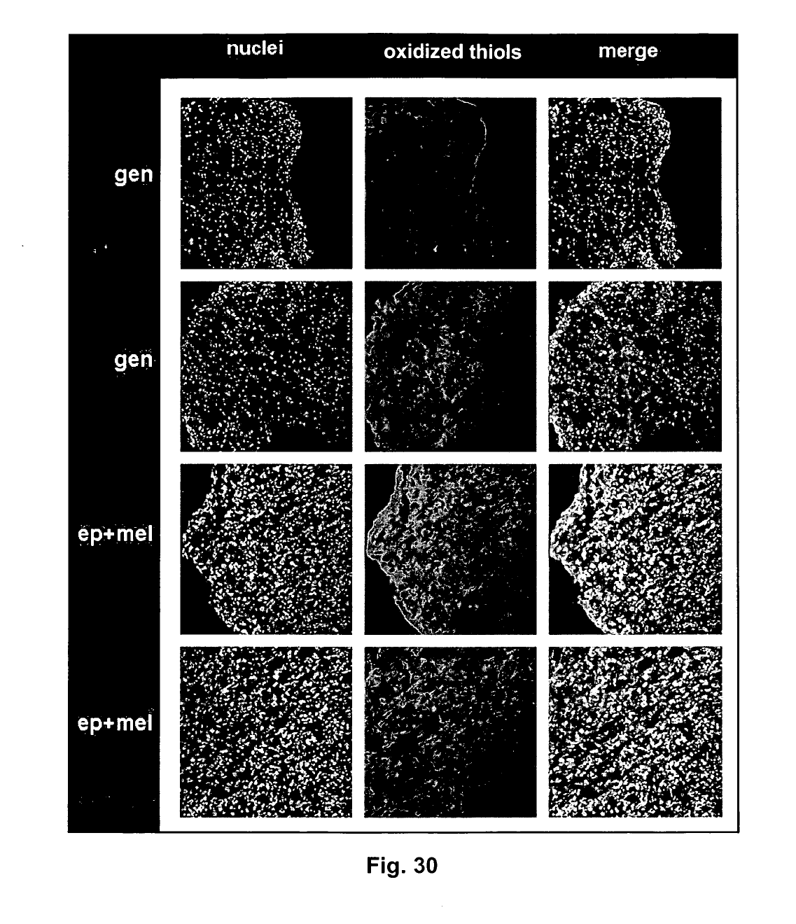

Duplicate experiments were conducted for various experimental conditions. FIG. 28 shows fluorescence microphotographs of tumor tissues treated with oxaliplatin or ethyl pyruvate. FIG. 29 shows fluorescence microphotographs of tumor tissues treated with Ringer's solution or mel. FIG. 30 shows fluorescence microphotographs of tumor tissues treated with gentamycin or a combination of ethyl pyruvate and mel. FIG. 31 shows fluorescence microphotographs of skin tissue, tumor tissue treated with oxaliplatin and ethyl pyruvate, and tumor tissue treated with gentamycin and ethyl pyruvate.

### Example 9

### Testing the Effect of a Chemotherapeutic Agent on Abnormal Cellular Oxidation State

In addition to various fluorescence spectroscopy methods, infrared analysis can be used to determine the cellular oxidation state. This method can be easily adapted to other dyes with different spectral properties. For example, dyes with excitation spectra in the red or far-red field, quantum dots, nanoparticles, or chemiluminescence probes can be used. By using different dyes with different spectral properties, a broader range of chemical and biochemical information can be obtained for the tissue sample. Detection in the red or far-red field eliminates the background noise from to UV-excitable molecules, such as amino acid residues with phenyl rings (e.g. tyrosine residues), plastics, and membranes.

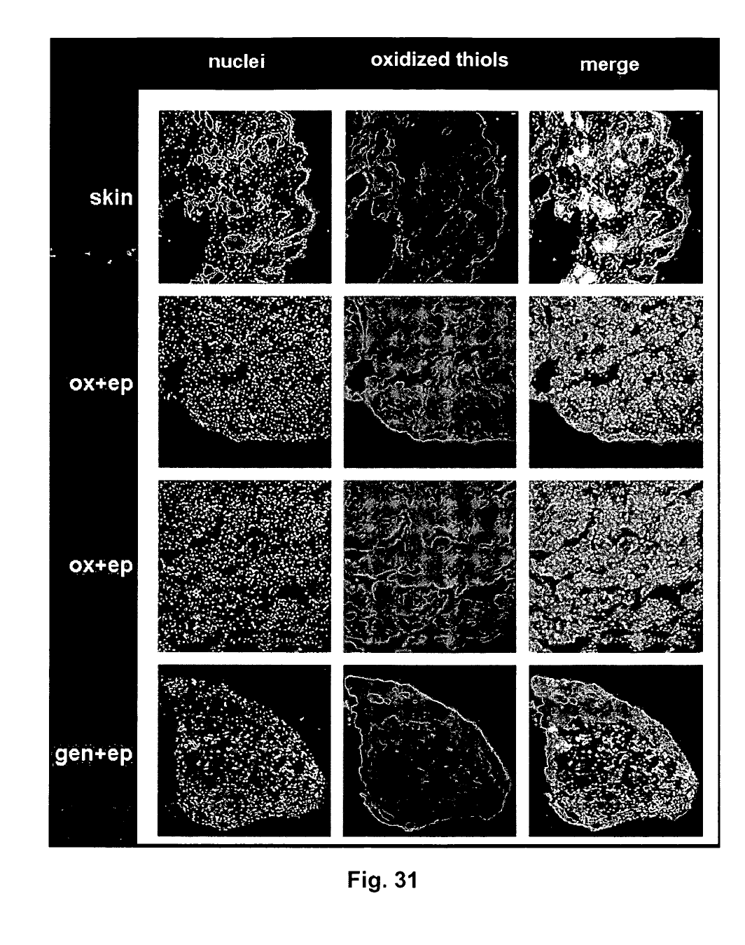

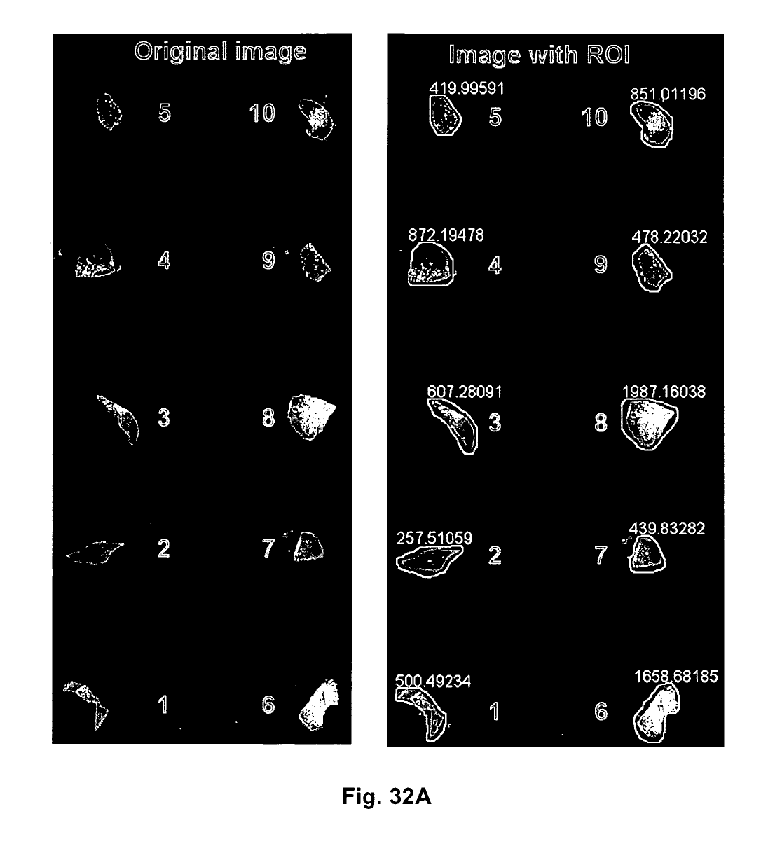

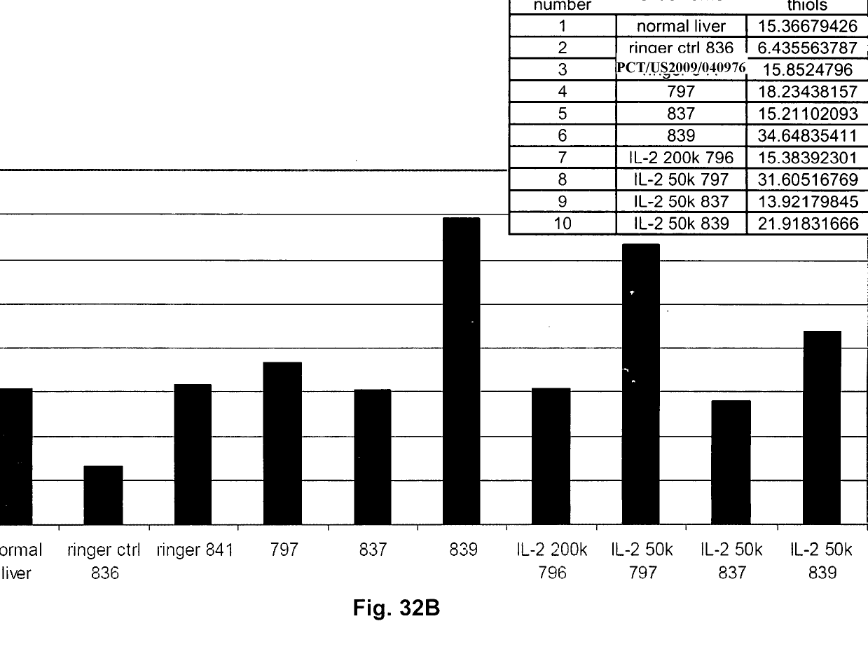

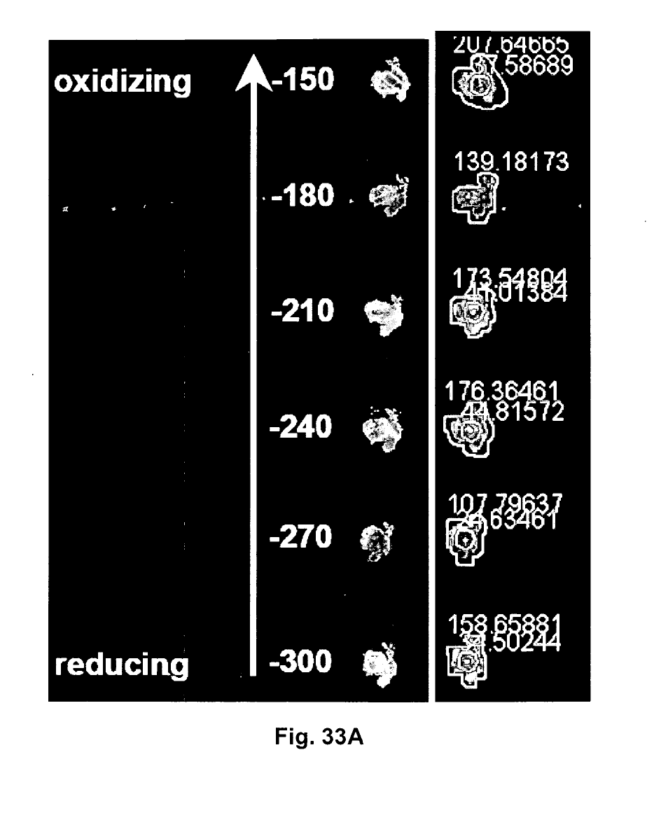

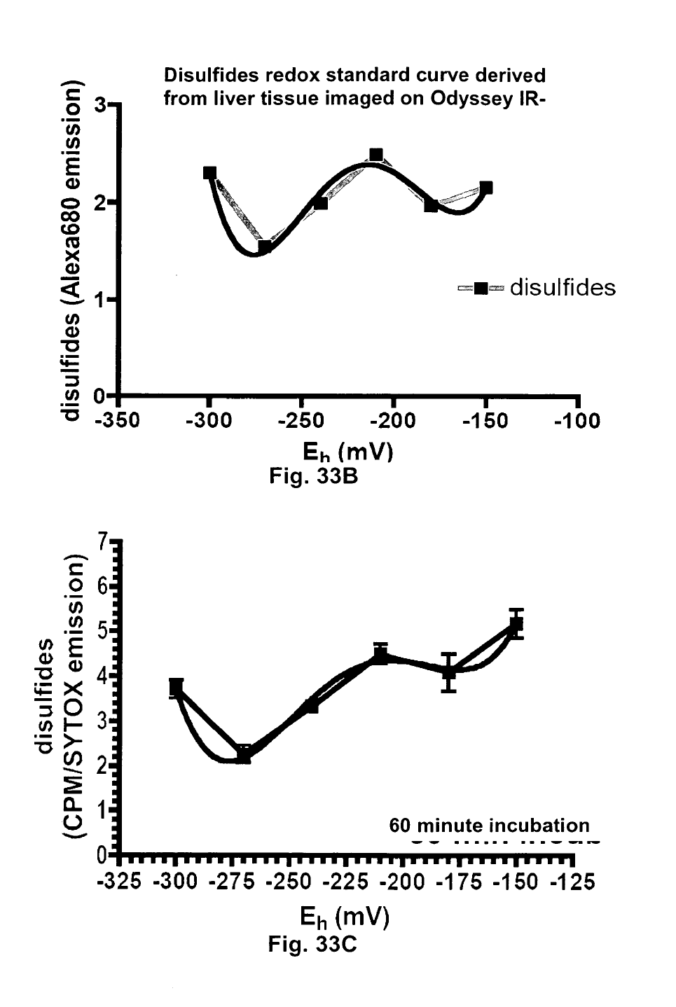

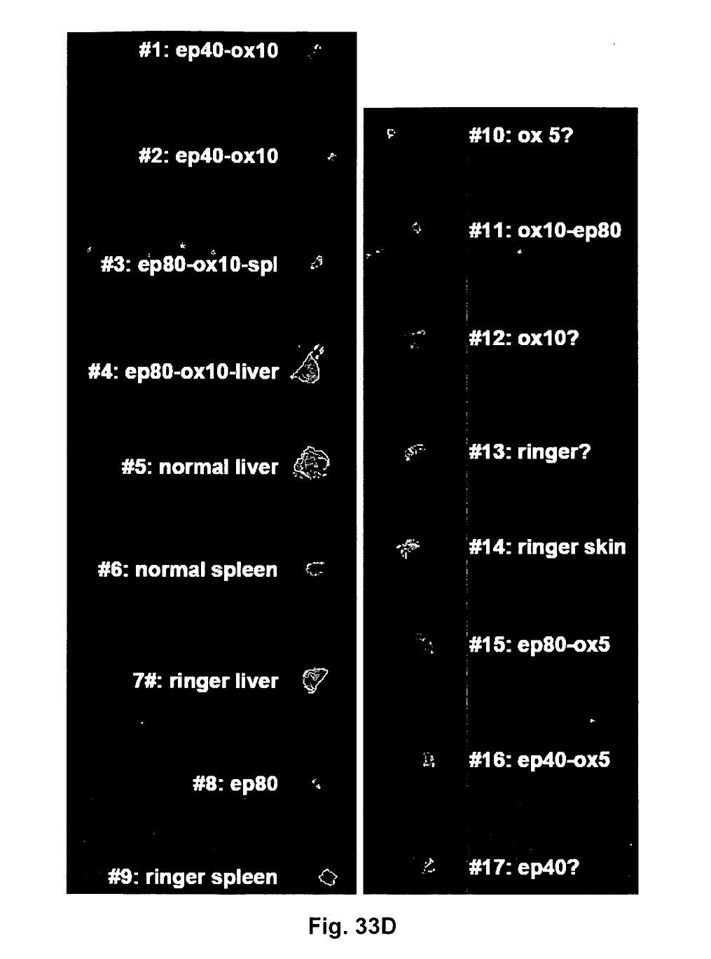

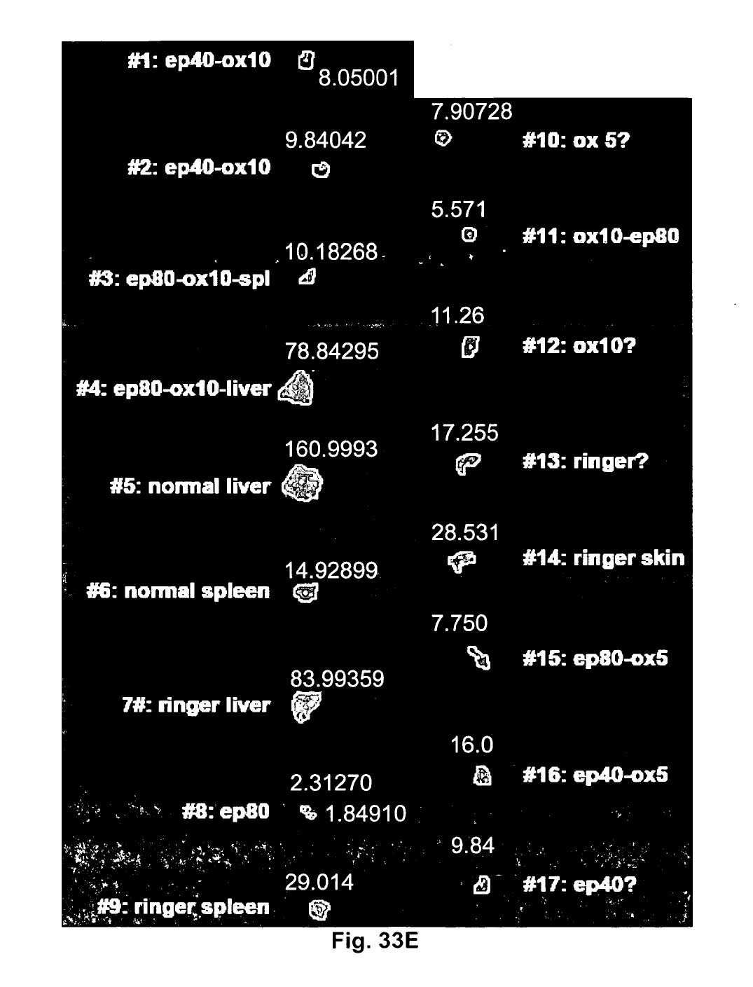

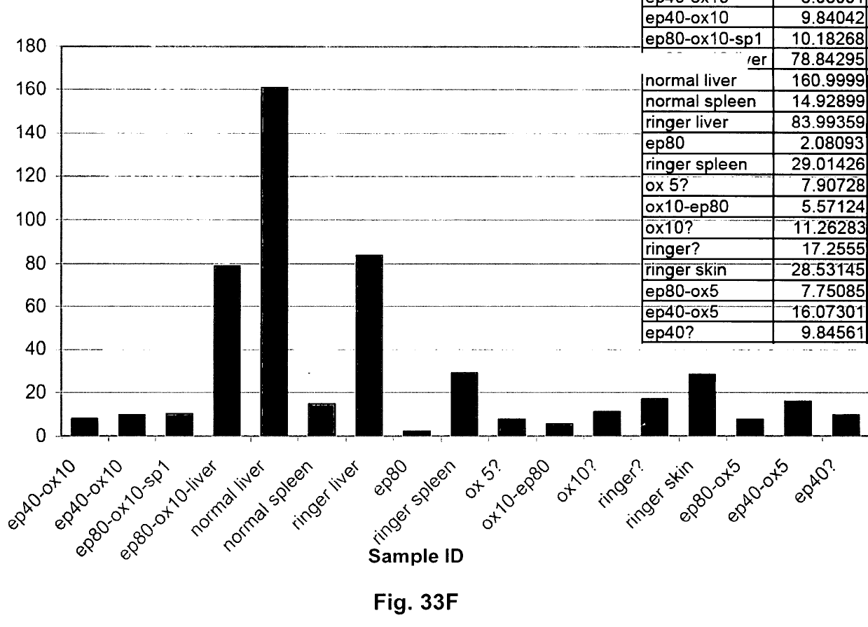

FIG. 32A shows infrared scanner images of liver tissue samples (labeled from 1 to 10) treated with various agents, where FIG. 32B shows the levels of oxidized thiols in samples that were analyzed using infrared scanner images. The liver tissue samples were treated as described in the table of the next FIG. 33A.

FIG. 33A shows infrared scanner images of tissue samples incubated in redox buffer with a redox potential from −300 mV to −150 mV. To quantify data obtained from infrared scanner images, integrated intensity is determined within the region of interest (ROI). FIG. 33B shows the integrated intensity of Alexa 680 emission (maximum emission at 702 nm) as a function of redox potential for liver tissue imaged with an infrared scanner. FIG. 33C is a graph shows CPM/SYTOX emission as a function of redox potential for SH-SY5Y human neuroblastoma cells imaged using fluorescence microscopy. The redox standard curve is reproducible in different specimens labeled with different fluorophores and analyzed with different instruments.

FIG. 33D shows infrared scanner images of different tissue samples treated with various agents (labeled from #1 to #17), where FIG. 33E shows these images with the region of interest (“ROI”) outlined in blue and integrated intensity obtained within the ROI. FIG. 33F shows levels of disulfides in samples that were analyzed using infrared scanner images. The table shows the sample ID and levels of disulfides.

We claim:

1. A method of labeling a protein, comprising labeling the protein with an oxidation labeling reagent comprising a carbonyl-reactive group and a first member of a fluorescence resonance energy transfer (FRET) pair; and further labeling the protein with a binding reagent comprising a second member of the FRET pair.

2. The method of claim 1, wherein the FRET pair comprises one of the pairs:
CPM/Alexa488 and CPM/FITC and FITC/Cy3.

3. The method of claim 1, wherein the carbonyl reactive group is a hydrazine derivative.

4. The method of claim 3, wherein the hydrazine derivative is one of hydrazine and dinitrophenyl hydrazine.

5. The method of claim 1, wherein the oxidation labeling reagent is fluorescein-conjugated hydrazide.

6. The method of claim 1 wherein a signal from FRET is detected.

7. The method of claim 6, wherein the signal is detected by one of fluorescent imaging, spectrophotometry, and fluorescent-activated cell sorting.

8. The method of claim 1, wherein the protein is selected from the group consisting of thioredoxin, HSP-60, Ask, PP2a, PTEN, cdc25, AP-1 NF-κB, NMDAR, RyR, respiratory complex I, proteins comprising the permeability transition pore, HMGB1, glutathione-S transferase, thioredoxin, glutaredoxin, and proteins involved in neurodegenerative diseases.

9. A method of labeling a protein, comprising:
alkylating free thiols in the protein with an alkylating agent;
reducing disulfide bonds the protein with a reducing agent;
labeling free thiols in the sample with an oxidation labeling reagent comprising an alkylating group and a first member of a fluorescence resonance energy transfer (FRET) pair; and further labeling the protein with a binding reagent comprising a second member of the FRET pair.

10. The method of claim 9, wherein the alkylating agent is one or more of a haloalkyl compound, an alpha-halocheto compound, a N-alkylmaleimide, an alkylmethanethiosulfonate, a p-hydroxymercurybenzoate, Ellman's reagent and a metal ion.

11. The method of claim 9, wherein the alkylating agent is one or more of monobromobimane, iodoacetic acid, iodoacetamide, N-ethylmaleimide, and methyl methanethiosulfonate, and mercury orange.

12. The method of claim 9, wherein the alkylating group is selected from the group consisting of a haloalkyl compound, an alpha-halocheto compound, a N-alkylmaleimide, an alkylmethanethiosulfonate, a p-hydroxymercurybenzoate, Ellman's reagent and a metal ion.

13. The method of claim 9, wherein the alkylating group is selected from the group consisting of monobromobimane, iodoacetic acid, iodoacetamide, N-ethylmaleimide, and methyl methanethiosulfonate, and mercury orange.

14. The method of claim 9, wherein a signal from FRET is detected by one of fluorescent imaging, spectrophotometry, and fluorescent-activated cell sorting.

15. The method of claim 10, wherein the reducing agent is selected from the group consisting of Cleland's reagent, DTT (dithiothreitol) or DTE (dithioerythriol); 2-mercaptoethanol (β-mercaptoethanol); 2-mercaptoethylamine; trivalent phosphines, TBP (tributylphosphine), and TCEP (tris(2-carboxyethyl)phosphine, and any combination thereof.

16. The method of claim 9, wherein the FRET pair comprises one of the pairs:
CPM/Alexa488 and CPM/FITC and FITC/Cy3.

17. A method of detecting a cellular oxidation state comprising, labeling a cellular protein with an oxidation labeling reagent comprising a carbonyl-reactive group and a first member of a fluorescence resonance energy transfer (FRET) pair; and further labeling the cellular protein with a binding reagent comprising a second member of the FRET pair.

18. The method of claim 17, wherein the cellular oxidation state is indicative of a disease or exposure to a toxic compound in a subject.

19. A method of screening compounds comprising:
contacting one or more cells having a cellular oxidation level with a compound;
detecting the presence of oxidized thiol or carbonyl groups, thereby assessing the oxidation level of the cells; and,
comparing the oxidation level of the cells treated with the compound with the oxidation level of the cells untreated with the compound, wherein a change in the oxidation level in the cells treated with the compound as compared to the oxidation level of the cells untreated with the compound is indicative of the ability of the compound to alter the oxidation level in the cells.

20. The method of claim 19, wherein the oxidation level is caused by a pathology selected from the group consisting of disease, cancer, genetic defects and toxin exposure.

21. The method of claim 20, wherein the oxidation state is caused by a toxin.

22. The method of claim 21, wherein the toxin is a pesticide.

23. The method of claim 22, wherein the pesticide is one or rotenone and paraquat.

24. A kit comprising an oxidation labeling reagent having a carbonyl-reactive group and a first member of a fluorescence resonance energy transfer (FRET) pair; and a binding reagent comprising a second member of the FRET pair.
